(19) United States
(12) Patent Application Publication
(10) Pub. No.: US 2004/0121022 A1
(43) Pub. Date: Jun. 24, 2004

(54) Culture media and methods of making and using culture media

(76) Inventors: William D. Holloway JR., Carlsbad, CA (US); Michael A. Holloway, Escondido, CA (US); Nikolai Tankovich, San Diego, CA (US); Eugene Baranov, San Diego, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 10/393,910

(22) Filed: Mar. 20, 2003

Related U.S. Application Data
(63) Continuation-in-part of application No. 09/698,537, filed on Oct. 26, 2000, now Pat. No. 6,521,248.
(60) Provisional application No. 60/161,546, filed on Oct. 26, 1999.

(51) Int. Cl. C12N 1/20; C12N 5/02; A61K 33/00

(52) U.S. Cl. 424/600; 435/404; 435/253.6

(57) ABSTRACT

Micro-clustered liquids, methods of manufacture and use. Culture media and cultures comprising micro-clustered water; use of micro-clustered culture media and cultures for cell, tissue and organ maintenance and growth; use in microbial biotechnology.

### RELATED APPLICATIONS

This application is a continuation-in-part U.S. patent application Ser. No. 09/698,537, filed Oct. 26, 2000 (and based on provisional application No. 60/161,546), which issued as U.S. Pat. No. 6,521,248, Feb. 18, 2003. This aforementioned applications are incorporated herein by reference in their entirety and for all purposes.

### FIELD OF THE INVENTION

The invention relates generally to micro-cluster liquids and methods of making and using them. The present invention provides a process of making micro-cluster liquid and methods of use thereof.

### BACKGROUND OF THE INVENTION

Water is composed of individual H₂O molecules that may bond with each other through hydrogen bonding to form clusters that have been characterized as five species: un-bonded molecules, tetrahedral hydrogen bonded molecules comprised of five (5) H₂O molecules in a quasi-tetrahedral arrangement and surface connected molecules connected to the clusters by 1, 2 or 3 hydrogen bonds, (U.S. Pat. No. 5,711,950 Lorenzen; Lee H.). These clusters can then form larger arrays consisting of varying amounts of these micro-cluster molecules with weak long distance van der Waals attraction forces holding the arrays together by one or more of such forces as; (1) dipole-dipole interaction, i.e., electrostatic attraction between two molecules with permanent dipole moments; (2) dipole-induced dipole interactions in which the dipole of one molecule polarizes a neighboring molecule; and (3) dispersion forces arising because of small instantaneous dipoles in atoms. Under normal conditions the tetrahedral micro-clusters are unstable and reform into larger arrays from agitation, which impart London Forces to overcome the van der Waals repulsion forces. Dispersive forces arise from the relative position and motion of two water molecules when these molecules approach one another and results in a distortion of their individual envelopes of intra-atomic molecular orbital configurations. Each molecule resists this distortion resulting in an increased force opposing the continued distortion, until a point of proximity is reached where London Inductive Forces come into effect. If the velocities of these molecules are sufficiently high enough to allow them to approach one another at a distance equal to van der Waals radii, the water molecules combine.

There is currently a need for a process whereby large molecular arrays of liquids can be advantageously fractionated. Furthermore, there is a desire for smaller molecular (e.g., micro-clusters) of water for consumption, medicinal and chemical processes.

### SUMMARY OF THE INVENTION

The inventors have discovered that liquids, which form large molecular arrays, such as through various electrostatic and van der Waal forces (e.g., water), can be disrupted through cavitation into fractionated or micro-cluster molecules (e.g., theoretical tetrahedral micro-clusters of water). The inventors have further discovered a method for stabilizing newly created micro-clusters of water by utilizing van der Waals repulsion forces. The method involves cooling the micro-cluster water to a desired density, wherein the micro-cluster water may then be oxygenated. The micro-cluster water is bottled while still cold. In addition, by overfilling the bottle and capping while the micro-cluster oxygenated water is dense (i.e., cold), the London forces are slowed down by reducing the agitation which might occur in a partially filled bottle while providing a partial pressure to the dissolved gases (e.g., oxygen) in solution thereby stabilizing the micro-clusters for about 6 to 9 months when stored at 40 to 70 degrees Fahrenheit.

The present invention provides a process for producing a micro-cluster liquid, such as water, comprising subjecting a liquid to cavitation such that dissolved entrained gases in the liquid form a plurality of cavitation bubbles; and subjecting the liquid containing the plurality of cavitation bubbles to a reduced pressure, wherein the reduction in pressure causes breakage of large liquid molecule matrices into smaller liquid molecule matrices. In another embodiment the liquid is substantially free of minerals and can be water which may also be substantially free of minerals. The embodiment provides for a process which is repeated until the water reaches about 140° C. (about 60° C.). The cavitation can be provided by subjecting the liquid to a first pressure followed by a rapid depressurization to a second pressure to form cavitation bubbles. The pressurization can be provided by a pump. In one embodiment the first pressure is about 55 psig to more than 120 psig. In another embodiment the second pressure is about atmospheric pressure. The embodiment can be carried out such that the pressure change caused the plurality of cavitation bubbles to implode or explode. The pressure change may be performed to create a plasma which dissociates the local atoms and reforms the atom at a different bond angle and strength. In another embodiment the liquid is cooled to about 4° C. to 15° C. Further embodiment comprises providing gas to the micro-cluster liquid, such as where the gas is oxygen. In a further embodiment the oxygen is provided for about 5 to about 15 minutes. In a further embodiment the invention provides a process for producing a micro-cluster liquid, comprising subjecting a liquid to a pressure sufficient to pressurize the liquid; emitting the pressurized liquid such that a continuous stream of liquid is created; subjecting the continuous stream of liquid to a multiple rotational vortex having a partial vacuum pressure such that dissolved and entrained gases in the liquid form a plurality of cavitation bubbles; and subjecting the liquid containing the plurality of cavitation bubbles to a reduced pressure, wherein the plurality of cavitation bubbles implode or explode causing shockwaves that break large liquid molecule matrices into smaller liquid molecule matrices. In a further embodiment the liquid is substantially free of minerals and in an additional embodiment the liquid is water, preferably substantially free of minerals. The invention provides that the process can be repeated until the water reaches about 140° F. (about 60° C.). In another embodiment the cavitation is provided by subjecting the liquid to a first pressure followed by a rapid depressurization to a second pressure to form cavitation bubbles. Further the invention provides that the pressurization is provided by a pump. In a further embodiment the first pressure is about 55 psig to more than 120 psig and, in another embodiment the second pressure is about atmospheric pressure, including embodiments where the second pressure is less than 5 psig. The invention also provides for micro-cluster liquid where the pressure change causes the plurality of cavitation bubbles to implode or explode. In a further embodiment, the pressure change creates a plasma which dissociates the local atoms and reforms the atoms at a different bond angle and strength. The invention also provides a process where the liquid is cooled to about 4° C. to 15° C. In another embodiment, the invention provides subjecting a gas to the micro-cluster liquid. Preferably, the gas is oxygen, especially oxygen administered for about 5 to 15 minutes and more preferably at pressure from about 15 to 20 psig.

The present invention also provides for a composition comprising a micro-cluster water produced according to the procedures noted above.

Still another aspect of the invention is a micro-cluster water which has any or all of the properties of a conductivity of about 3.0 to 4.0 μmhos/cm, a FTIR spectrophotometric pattern with a major sharp feature at about 2650 wave numbers, a vapor pressure between about 40° C. and 70° C. as determined by thermogravimetric analysis, and an ¹⁷O NMR peak shift of at least about +30 Hertz, preferably at least about +40 Hertz relative to reverse osmosis water.

The present invention further provides for the use of the micro-cluster water of the invention for such purposes as modulating cellular performance and lowering free radical levels in cells by contacting the cell with the micro-cluster water.

The present invention further provides a delivery system comprising a micro-cluster water (e.g., an oxygenated microcluster water) and an agent, such as a nutritional agent, a medication, and the like.

Further, the micro-cluster water of the invention can be used to remove stains from fabrics by contacting the fabric with the micro-cluster water.

The details of one or more embodiments of the invention are set forth in the accompanying drawings and the description below. Other features, objects, and advantages of the invention will be apparent from the description and drawings, and from the claims.

All publications, patents and patent applications cited herein are hereby expressly incorporated by reference for all purposes.

### BRIEF DESCRIPTION OF THE DRAWINGS

FIG. 1 shows a water molecule and the resulting net dipole moment.

FIG. 2 shows a large array of water molecules.

FIG. 3 shows a micro-cluster of water having 5 water molecules forming a tetrahedral shape.

FIG. 4 shows an example of a device useful in creating cavitation in a liquid. The device provides inlets for a liquid, wherein the liquid is then subjected to multiple rotational vortexes reaching partial vacuum pressures of about 27″ Hg. The liquid then exits the device at point A through an acceleration tube into a chamber less than the pressure within the device (e.g., about atmospheric pressure).

FIG. 5 shows FTIR spectra for RO water (FIG. 5(a)) and processed micro-cluster water (FIG. 5(b)).

FIG. 6 shows TGA plots for RO water and oxygenated micro-cluster water.

FIG. 7 shows NMR spectra for RO water (FIG. 7(a)), micro-cluster water without oxygenation (FIG. 7(b)) and micro-cluster water with oxygenation (FIG. 7(c)).

FIG. 8 shows a schematic illustration of a device for Raman spectroscopy.

FIG. 9 shows the effects of micro-clustered cell culture medium on macrophage plasma membranes.

FIG. 10 shows the effects of micro-clustered cell culture medium on intracellular pH.

FIG. 11 shows the effects of micro-clustered cell culture medium on the viability of 293T cells.

FIGS. 12a and 12b show the effects of micro-clustered water on growth and transfection of two types of human cells.

FIG. 13 shows the effects of micro-clustered water on the expression profiles of dendritic cell markers.

FIG. 14 shows the effects of micro-clustered water on the functional state of brain tissue perfused with micro-clustered medium.

### DESCRIPTION OF THE PREFERRED EMBODIMENTS

Liquids, including for example, alcohols, water, fuels and combinations thereof, are comprised of atoms and molecules having complex molecular arrangements. Many of these arrangements result in the formation of large molecular arrays of covalently bonded atoms having non-covalent interactions with adjacent molecules, which in turn interact via additional non-covalent interactions with yet other molecules. These large arrays, although stable, are not ideal for many applications due to their size. Accordingly it is desirable to create and provide liquids having smaller arrays by reducing the number of non-covalent interactions. These smaller molecules are better able to penetrate and react in biological and chemical systems. In addition, the smaller molecular arrays provide novel characteristics that are desirable.

As used herein, “covalent bonds” means bonds that result when atoms share electrons. The term “non-covalent bonds” or “non-covalent interactions” means bonds or interactions wherein electrons are not shared between atoms. Such non-covalent interactions include, for example, ionic (or electrovalent) bonds, formed by the transfer of one or more electrons from one atom to another to create ions, interactions resulting from dipole moments, hydrogen bonding, and van der Waals forces. Van der Waals forces are weak forces that act between non-polar molecules or between parts of the same molecule, thus bringing two groups together due to a temporary unsymmetrical distribution of electrons in one group, which induces an opposite polarity in the other. When the groups are brought closer than their van der Waals radii, the force between them becomes repulsive because their electron clouds begin to interpenetrate each other.

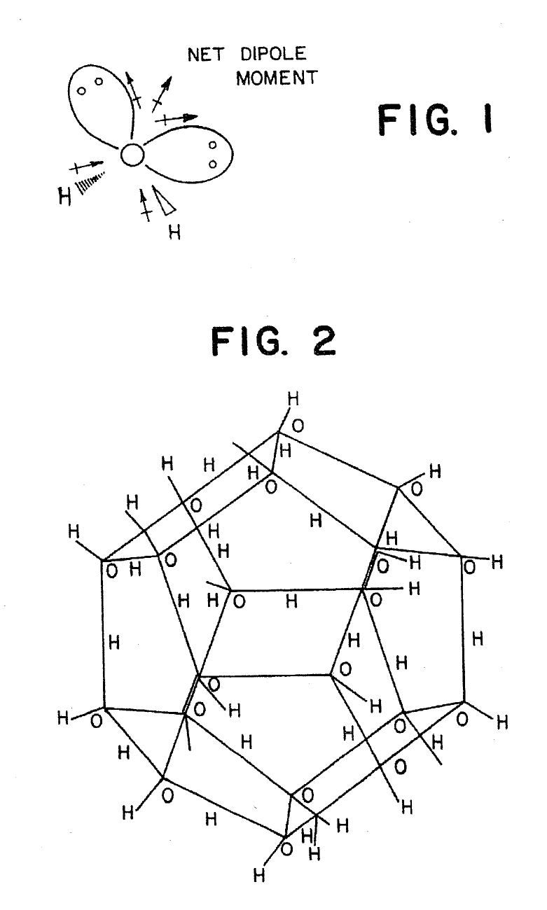

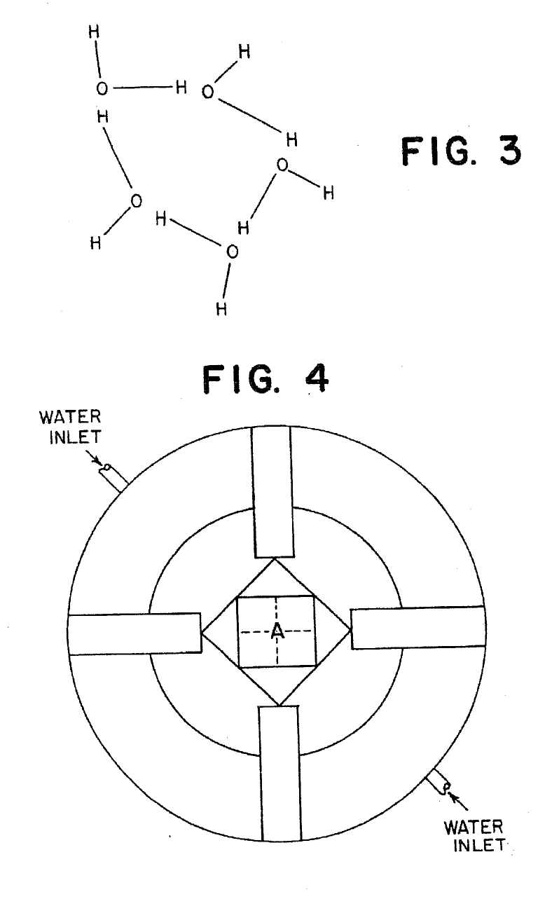

Numerous liquids are applicable to the techniques described herein. Such liquids include water; alcohols, petroleum and fuels. Liquids, such as water, are molecules comprising one or more basic elements or atoms (e.g., hydrogen and oxygen). The interaction of the atoms through covalent bonds and molecular charges form molecules. A molecule of water has an angular or bent geometry. The H—O—H bond angle in a molecule of water is about 104.5° to 105°. The net dipole moment of a molecule of water is depicted in FIG. 1. This dipole moment creates electrostatic forces that allow for the attraction of other molecules of water. Recent studies by Pugliano et al., (Science, 257:1937, 1992) have suggested the relationship and complex interactions of water molecules. These studies have revealed that hydrogen bonding and oxygen-oxygen interactions play a major role in creating large clusters of water molecules. Substantially purified water forms complex structures comprising multiple water molecules each interacting with an adjacent water molecule (as depicted in FIG. 2) to form large arrays. These large arrays are formed based upon, for example, non-covalent interactions such as hydrogen bond formation and as a result of the dipole moment of the molecule. Although highly stable, these large molecules have been suggested to be detrimental in various chemical and biological reactions. Accordingly, in one embodiment, the present invention provides a method of forming fractionized or micro-cluster water as depicted in FIG. 3 having as few as about 5 molecules of water.

The present invention provides small micro-cluster liquids (e.g., micro-cluster water molecules) a method for manufacturing fractionized or micro-cluster water and methods of use in the treatment of various biological conditions.

Accordingly, the present invention provides a method for manufacturing fractionized or micro-cluster liquids (e.g., water) comprising pressurizing a starting liquid to a first pressure followed by rapid depressurization to a second pressure to create a partial vacuum pressure that results in release of entrained gases and the formation of cavitation bubbles. The thermo-physical reactions provided by the implosion and explosion of the cavitation bubbles results in an increase in heat and the breaking of non-covalent interactions holding large liquid arrays together. This process can be repeated until a desired physical-chemical trait of the fractionized liquid is obtained. Where the liquid is water, the process is repeated until the water temperature reaches about 140° F. (about 60° C.). The resulting smaller or fractionized liquid is cooled under conditions that prevent reformation of the large arrays. As used herein, “water” or “a starting water” includes tap water, natural mineral water, and processed water such as purified water.

Any number of techniques known to those of skill in the art can be used to create cavitation in a liquid so long as the cavitating source is suitable to generate sufficient energy to break the large arrays. The acoustical energy produced by the cavitation provides energy to break the large liquid arrays into smaller liquid clusters. For example, the use of acoustical transducers may be utilized to provide the required cavitation source. In addition, cavitation can be induced by forcing the liquid through a tube having a constriction in its length to generate a high pressure before the constriction, which is rapidly depressurized following the constriction. Another example, includes forcing a liquid through a pump in reverse direction through a rotational volute.

In one embodiment, a liquid to be fractionized is pressurized into a rotational volute to create a vortex that reaches partial vacuum pressures releasing entrained gases as cavitation bubbles when the rotational vortex exits through a tapered nozzle at or close to atmospheric pressure. This sudden pressurization and decompression causes implosion and explosion of cavitation bubbles that create acoustical energy shockwaves. These shockwaves break the covalent and non-covalent bonds on the large liquid arrays, break the weak array bonds, and form micro-cluster or fractionized liquid consisting of, for example, about five (5) H₂O molecules in a quasi tetrahedral arrangement (as depicted in FIG. 3), and impart an electron charge to the micro-cluster liquid thus producing electrolyte properties in the liquid. The micro-cluster liquid is recycled until desired number of micro-cluster liquid molecules are formed to reach a given surface tension and electron charge, as determined by the temperature rise of the liquid over time as cavitation bubbles impart kinetic heat to the processed liquid. Once the desired surface tension and electron charge are reached the micro-cluster liquid is cooled until liquid density increases. The desired surface tension and electron charge can be measured in any number of ways, but is preferably detected by temperature. Once the liquid reaches a desired density, typically at about 4 to 15° C., a gas, such as, for example, molecular oxygen, can be introduced for a sufficient amount of time to attain the desired quantity of oxygen in the micro-cluster liquid. The micro-cluster liquid is then aliquoted into a container or bottle, preferably filled to maximum capacity, and capped while the gassed micro-cluster liquid is still cool, so as to provide a partial pressure to the gassed micro-cluster liquid as the temperature reaches room temperature. This enables larger quantities of dissolved gas to be maintained in solution due to increased partial pressure on the bottles contents.

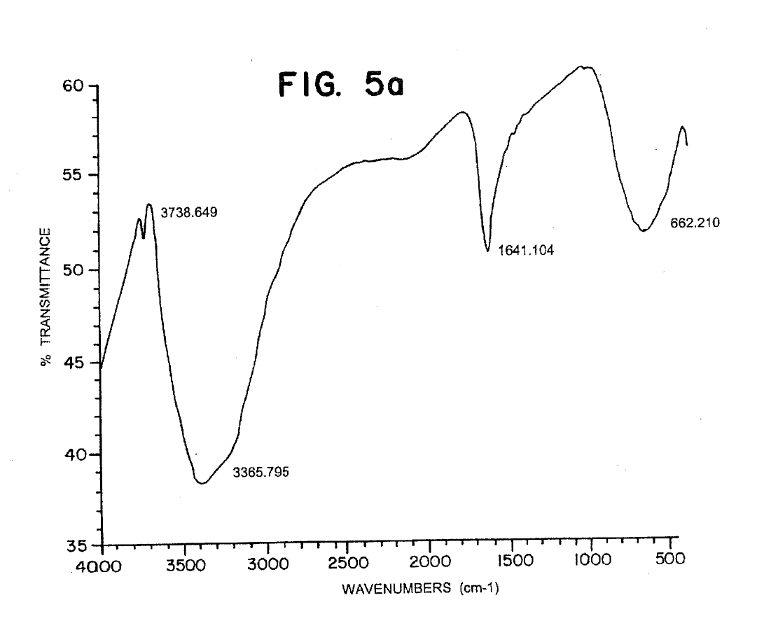

The present invention provides a method for making a micro-cluster or fractionized water or liquid, for ease of explanation water will be used as the liquid being described, however any type liquid may be substituted for water. A starting water such as, for a example, purified or distilled water is preferably used as a base material since it is relatively free of mineral content. The water is then placed into a food grade stainless steel tank for processing. By subjecting the starting water to a pump capable of supplying a continuous pressure of between about 55 and 120 psig or higher a continuous stream of water is created. This stream of water is then applied to a suitable device (see for example FIG. 4) capable of establishing a multiple rotational vortex reaching partial vacuum pressures of about 27″ Hg, thereby reaching the vapor pressure of dissolved entrained gases in the water. These gases form cavitation bubbles that travel down multiple acceleration tubes exiting into a common chamber at or close to atmospheric pressure. The resultant shock waves produced by the imploding and exploding cavitation bubbles breaks the large water arrays into smaller water molecules by repeated re-circulation of the water. The recycling of the water creates increases results in an increase in temperature of the water. The heat produced by the imploding and exploding cavitation bubbles release energy as seen in sonoluminescence, in which the temperature of sonoluminance bubbles are estimated to range from 10 to 100 eV or 2,042.033 degrees Fahrenheit at 19,743,336 atmospheres. However the heat created is at a sub micron size and is rapidly absorbed by the surrounding water imparting its kinetic energy. The inventors have determined that the breaking of these large arrays into smaller water molecules can be manipulated through a sinusoidal wave utilizing cavitation, and by monitoring the rise in temperature one can adjust the osmotic pressure and surface tension of the water under treatment. The inventors have determined that the ideal temperature for oxygenated micro-cluster water (Penta-hydrate™) is about 140 degrees F. (about 60° C.). This can be accomplished by using four opposing vortex volutes with a 6-degree acceleration tube exiting into a common chamber at or close to atmospheric pressure, less than 5 pounds backpressure.

As mentioned above, the inventors have also discovered that liquids undergo a sinusoidal fluctuation in heat/temperature under the process described herein. Depending upon the desired physical-chemical traits, the process is repeated until a desired point in the sinusoidal curve is established at which point the liquid is collected and cooled under, conditions to inhibit the formation of large molecular arrays. For example, and not by way of limitation, the inventors have discovered that water processed according to the methods described herein undergoes a sinusoidal heating process. During the production of this water a high negative charge is created and imparted to the water. Voltages of −350 mV to −1 volt have been measured with a superimposed sinusoidal wave with a frequency of 800 cycles or higher depending on operating pressures and subsequent water velocities. The inventors have found that the third sinusoidal peak in temperature provides an optimal number of micro-cluster structures for water. Although the inventors are under no duty to provide the mechanism or theory of action, it is believed that the high negative ion production serves as a ready source of donor electrons to act as antioxidants when consumed and further act to stabilize the water micro-clusters and help prevent reformation of the large arrays by aligning the water molecules exposed to the electrostatic field of the negative charge. While not wanting to be bound to a particular theory, it is believed that the high temperatures achieved during cavitation may form a plasma in the water which dissociates the H₂O atoms and which then reform at a different bond association, as evidenced by the FTIR and NMR test data, to generate a different structure.

It will be recognized by those skilled in the art that the water of the present invention can be further modified in any number of ways. For example, following formation of the micro-cluster water, the water may be oxygenated as described herein, further purified, flavored, distilled, irradiated, or any number of further modifications known in the art and which will become apparent depending on the final use of the water.

In another embodiment, the present invention provides methods of modulating the cellular performance of a tissue or subject. The micro-cluster water (e.g., oxygenated microcluster water) can be designed as a delivery system to deliver hydration, oxygenation, nutrition, medications and increasing overall cellular performance and exchanging liquids in the cell and removing edema. Tests accomplished utilizing an RJL Systems Bio-Electrical Impedance Analyzer model BIA101 Body Composition Analysis System™ demonstrated substantial intracellular and extracellular hydration, changes in as little as 5 minutes. Tests were accomplished on a 58-year-old male 71.5″ in height 269 lbs, obese body type. Baseline readings were taken with Bio-Electrical Impedance Analyzer™ as listed below.

As described in the Examples below it is contemplated that the micro-cluster water of the present invention provides beneficial effects upon consumption by a subject. The subject can be any mammal (e.g, equine, bovine, porcine, murine, feline, canine) and is preferably human. The dosage of the micro-cluster water or oxygenated micro-cluster water (Penta-hydrate™) will depend upon many factors recognized in the art, which are commonly modified and adjusted. Such factors include, age, weight, activity, dehydration, body fat, etc. Typically 0.5 liters of the oxygenated micro-cluster water of the invention provide beneficial results. In addition, it is contemplated that the micro-cluster water of the invention may be administered in any number of ways known in the art, including, for example, orally and intravenously alone or mixed with other agents, compounds and chemicals. It is also contemplated that the water of the invention may be useful to irrigate wounds or at the site of a surgical incision. The water of the invention can have use in the treatment of infections, for example, infections by anaerobic organisms may be beneficially treated with the micro-cluster water (e.g., oxygenated microcluster water).

In another embodiment, the micro-cluster water of the invention can be used to lower free radical levels and, thereby, inhibit free radical damage in cells.

In still another embodiment the micro-cluster water of the invention can be used to remove stains from fabrics, such as cotton.

The following examples are meant to illustrate but no limit the present invention. Equivalents of the following examples will be recognized by those skilled in the art and are encompassed by the present disclosure.

### EXAMPLE 1

How to Make Micro-Cluster Water

Described below is one example of a method for making micro-cluster liquids. Those skilled in the art will recognize alternative equivalents that are encompassed by the present invention. Accordingly, the following examples is not to be construed to limit the present invention but are provided as an exemplary method for better understanding of the invention.

325 gallons of steam distilled water from Culligan Water or purified in 5 gallon bottles at a temperature about 29 degrees C. ambient temperature, was placed in a 316 stainless steel non-pressurized tank with a removable top for treatment. The tank was connected by bottom feed 2 ¼″ 316 stainless steel pipe that is reduced to 1″ NPT into a 20″ U.S. filter housing containing a 5 micron fiber filter, the filter serves to remove any contaminants that may be in the water. Output of the 20″ filter is connected to a Teel model 1 V458 316 stainless steel Gear pump driven by a 3HP 1740 RPM 3 phase electric motor by direct drive. Output of the gear pump 1″ NPT was directed to a cavitation device via 1″ 316 stainless steel pipe fitted with a 1″ stainless steel ball valve used for isolation only and pasta pressure gauge. Output of the pump delivers a continuous pressure of 65 psig to the cavitation device.

The cavitation device was composed of four small inverted pump volutes made of Teflon without impellers, housed in a 316 stainless steel pipe housing that are tangentially fed by a common water source fed by the 1 V458 Gear pump at 65 psig, through a ¼″ hole that would normally be used as the discharge of a pump, but are utilized as the input for the purpose of establishing a rotational vortex. The water entering the four volutes is directed in a circle 360 degrees and discharged through what would normally be the suction side of a pump by the means of an 1″ long acceleration tube with a ⅜″ discharge hole, comprising what would normally be the suction side of a pump volute but in this case is utilized as the discharge side of the device. The four reverse fed volutes establish rotational vortexes that spin the water one 360 degree rotation and then discharge the water down the 5 degree decreasing angle from center line, acceleration tubes discharging the water into a common chamber at or close to atmospheric pressure. The common chamber was connected to a 1″ stainless steel discharge line that fed back into the top of the 325-gallon tank containing the distilled water. At this point the water made one treatment trip through the device.

The process listed above is repeated continuously until the energy created by the implosions and explosions of the cavitation (e.g, due to the acoustical energy) have imparted its kinetic heat into the water and the water is at about 60 degrees Celsius.

Although the inventors are under no duty to explain the theory of the invention, the inventors provide the following theory in the way of explanation and are not to be bound by this theory. The inventors believe that the acoustical energy created by the cavitation brakes the static electric bonds holding a single tetrahedral Micro-Clusters of five H₂O molecules together in larger arrays, thus decreasing their size and/or create a localized plasma in the water restructuring the normal bond angles into a different structure of water.

The temperature was detected by a hand held infrared thermal detector through a stainless steel thermo well. Other methods of assessing the temperature will be recognized by those of skill in the art. Once the temperature of 60 degrees C. has been reached the pump motor is secured and the water is left to cool. An 8 foot by 8 foot insulated room fitted with a 5,000 Btu. air conditioner is used to expedite cooling, but this is not required. It is important that the processed water not be agitated for cooling it should be moved as little as possible.

A cooling temperature of 4 degrees C. can be used, however 15 degrees C. is sufficient and will vary depending upon the quantity of water being cooled. Once sufficiently cooled to about 4 to 15 degrees C. the water can be oxygenated.

Once the water is cooled to desired temperature, the processed water is removed from the 325 gallon stainless steel tank into 5-gallon polycarbonate bottles for oxygenation.

Oxygenation is accomplished by applying gas O₂ at a pressure of 20 psig fed through a ¼″ ID plastic line fitted with a plastic air diffuser utilized to make fine air bubbles (e.g., Lee's Catalog number 12522). The plastic tube is run through a screw on lid of the 5 gallon bottle until it reaches the bottom of the bottle. The line is fitted with the air diffuser at its discharge end. The Oxygen is applied at 20 psig flowing pressure to insure a good visual flow of oxygen bubbles. In one embodiment (Penta-hydrate™) the water is oxygenated for about five minutes and in another embodiment (Penta-hydrate Pro™) the water is oxygenated for about ten minutes.

Immediately after oxygenation the water is bottled in 500 ml PET bottles, filled to overflowing and capped with a pressure seal type plastic cap with inserted seal gasket. In one embodiment, the 0.5 L bottle is over filled so when the temperature of the water increases to room temperature it will self pressurize the bottle retaining a greater concentration of dissolved oxygen at partial pressure. This step not only keeps more oxygen in a dissolved state but also for preventing excessive agitation of the water during shipping.

### EXAMPLE 2

The following are reports from individuals who used the water of the invention.

Elimination Of Edema:

Patient A: A 66-year-old Male presenting with (ALS) Amyothrophic Lateral Sclerosis (Lou Gherig's Disease) exhibited a shoulder hand syndrome with marked swelling of the left hand. This hand being the predominately affected limb. After consuming 500 ml of Penta-hydrate™ micro-cluster water the swelling of the left hand was dramatically reduced to normal state. Additional tests were accomplished over several weeks noting the same reduction of edema after consuming Penta-hydrate™ micro-cluster water. When Penta-hydrate™ was discontinued edema reoccurred overnight, upon consuming 500 ml of Penta-hydrate™ micro-cluster water edema was reduced within 4 to 6 hours.

Patient B: Is a 53 year old female with multijoint Acute Rheumatoid Arthritis of 6 year duration. She has been taking diuretics for dependent edema on a daily basis for 4 years. She began taking Penta-hydrate™ Micro-Cluster Water, 5 months ago in place of diuretics, consuming three (3) 500 ml bottles daily. Within one day the edema of the feet/legs and hands cleared. When Penta-hydrate™ was discontinued during a trip, the edema promptly returned. Upon resumption of Penta-hydrate™ Micro-Cluster Water the edema quickly cleared.

Increased Physical Endurance:

A 56-year-old woman diagnosed with “severe emphysema” and retired on full disability underwent experimental lung reduction surgery in December 1998 at St Elizabeth's Hospital in Boston. Each of the lungs upper lobes were removed and re-sectioned. While the surgery was deemed successful the patient had begun to deteriorate. The depression and loss of stamina was overcome by Oxy-Hi-drate Pro: A 2⅓ increase in endurance is usually seen in response to subject taking Penta-hydrate™ and is caused by increased delivery of hydration to the cells, which is the delivery system for increased oxygenation and cellular energy production. Tests on numerous test subjects show marked increase in cellular hydration within 10 minutes of consuming Penta-hydrate™ micro-cluster water.

Decreased Lactic Acid Soreness from Exercise:

The inventors have received reports of reduced or eliminated soreness caused by lactic acid buildup during exercise as well as increased endurance and performance after consuming Penta-hydrate™ micro-cluster water. This includes elderly fibromyalgia patients. Penta-hydrate™ micro-cluster is thought to delay or prevent the on set of anaerobic cellular function by increasing cellular water and oxygen exchange keeping the cells operating aerobic condition for a longer time period during strenuous exercise, thus preventing or delaying the buildup of lactic acid in the body.

Increased Athletic Performance:

Test accomplished on three high performance athletes have demonstrated a marked increase in overall performance.

A 29 year old male Tri-athlete competing in the 1999 Coronado Calif. 21^{St} annual Super Frog Half Iron Man Triathlon consumed (6) six 500 ml bottles of Penta-hydrate™ Micro-Cluster the day prior to the race and (6) six 500 ml bottles of Penta-hydrate™ during the race posted a finish time of 4:19:37 winning the overall male winner, finishing over 24 minutes ahead of the second place finisher in his age group and beating the combined time of the Navy SEAL Relay Team One's time of 4:26:09 which had a fresh man for each leg of the three events. Normally after such a demanding race this athlete would be extremely sore the next day, however drinking the Penta-hydrate™ Micro-Cluster Water he was not sore and competed in a 20 K cycle qualifier the following day. Subject Tri-Athlete has won numerous Triathlons' and qualified for the 1999 World-Championships in Australia.

A 39 year old male Tri-athlete competing in the San Diego Second Annual Duadrome World Championships on Aug. 8, 1999 at the Morley Field Velodrome. Subject athlete was pre hydrated with Penta-hydrate™ Micro-Cluster Water set a new world record winning the 35-39 age group division, beating his own best time by 26 seconds in the male relay division and the course record by 3 seconds

Both of the above Tri-athletes report dramatic increase in endurance and rapid recovery after strenuous exercise not experienced with conventional water and an ability to hydrate during the running portion of a triathlon, normally hydration is only accomplished during the cycling portion of a triathlon, due to normal water causing the subject to regurgitate, this problem is not encountered drinking Penta-hydrate™ Micro-Cluster Water due to its rapid absorption.

45-year-old woman TV 10 News anchor in San Diego, that also competes in rough ocean swimming. Consumed 500 ml of Penta-hydrate™ just prior to entering the water in a swim meet in Hawaii; won the gold medal in 45-year-old age division. Returned to San Diego and competed in the La Jolla rough water swim and won a gold medal. Next competed in the US Nationals held at Catalina Island in California and won the US National Gold Medal after drinking 500 ml of Penta-hydrate™ just prior to entering the water. She was not considered a contender for the Gold in the US Nationals.

Congestive Heart Failure:

The inventors have had several reports from subjects with congestive heart failure report ten minutes after consuming 500 ml of Penta-hydrate Pro™ their shortness of breath had gone away and their energy was increased.

Muscular Sclerosis MS:

A woman with Muscular Sclerosis was rushed to the hospital in San Antonio Tex. having passed out from severe dehydration. The MS subject drank×500 ml bottles of Penta-hydrate™ their and was re-hydrated.

Colds, Flu, Sinus Infections and Energy:

58-year-old male with loss of spleen and 20-year sufferer of fibromyalgia, suffered from chronic sinus infections and annual bouts of the flu and reoccurring bouts of pneumonia. He started drinking 6-500 ml bottles of Penta-hydrate™ Micro-Cluster Water per day 19 months ago. At that time he had a severe sinus infection that would have normally required antibiotics. While taking the Penta-hydrate™ Micro-Cluster Water, the sinus infection was cleared within three days and subject has not had a single sinus infection in 19 months. In addition he has not experienced any colds, flu or allergy conditions and is now for the first time in 20-years able to work with out fatigue.

Elimination of Edema:

In numerous test cases Penta-hydrate™ has eliminated edema in all test subjects from both chronic health conditions as well as surgically caused edema. In all cases edema was dramatically reduced after consuming as little as one 500 ml bottle of Penta-hydrate™ Micro-Cluster Water but no more than two 500 ml bottles were required. One such case was a middle-aged woman that had broken her forearm in two places. The forearm was in a cast and suffering severs edema, subject was given two 500 ml bottles of Penta-hydrate™ Micro-Cluster Water that she consumed from 3:00 pm until bedtime. Swelling was so bad that she could not insert a business card between her swollen arm and the cast. When she awoke at 7:00 am the next morning the swelling was reduced to where she was endanger of loosing the cast and had to return to the orthopedic surgeon to have the cast redone.

Liquid Nutritional Analyzer Results.

Liquid nutritional analyzer results utilizing a RJL Systems BIA100Q™ FDA registered analyzer for assessing cellular hydration and health. The following measurements were preformed on a 58 year-old male subject.

Time: 7:59 am Oct. 9, 1999 Baseline Test:

| | |
|---|---|
| Measured: Resistance: 413 ohms | Reactance: 53 ohms |
| Calculated: Impedance 416 ohms | Phase Angle: 7.3 degrees |
| Parallel Model: Resistance: 419.8 ohms | Capacitance: 973.0 pF |

| | | | | |
|---|---|---|---|---|
| Fluid Assessment: | | | | |
| Status: (Edema) | Results: | Percent: | Normal Range: | Deviation: |
| Total Body Water | 63.3 L | 52% (WT) | 40%-50% | +2 |
| Intracellular Water | 37.5 L | 59% (TBW) | 51%-60% | +0 |
| Extracellular Water | 25.8 L | 41% (TBW) | 39%-51% | +0 |

| | | |
|---|---|---|
| Nutrition Assessment: | | |
| Basal Metabolism | 2069 Kcal | |
| Body Cell Mass | 90.6 lbs. | 34% (WT) |
| Fat Free Mass | 190.2 lbs. | 71% |
| Fat | 78.8 lbs. | 29% |
| ECT | 99.6 lbs. | 52% |
| Impedance Index | 1437 Normal | |

Time: 8:02 am consumed 500 ml Penta-hydrate Pro ™

Time: 8:12 am Oct. 9, 1999

| | |
|---|---|
| Measured: Resistance: 436 ohms | Reactance: 57 ohms |
| Calculated: Impedance 439.7 ohms | Phase Angle: 7.4 degrees |
| Parallel Model: Resistance: 443.5 ohms | Capacitance: 938.4 pF |

| | | | | |
|---|---|---|---|---|
| Fluid Assessment: | | | | |
| Status: (Edema) | Results: | Percent: | Normal Range: | Deviation: |
| Total Body Water | 63.3 L | 51% (WT) | 40%-50% | +1 |
| Intracellular Water | 37.1 L | 60% (TBW) | 51%-60% | +0 |
| Extracellular Water | 25.2 L | 40% (TBW) | 39%-51% | +0 |

| | | |
|---|---|---|
| Nutrition Assessment: | | |
| Basal Metabolism | 2060 Kcal | |
| Body Cell Mass | 89.6 lbs. | 33% (WT) |
| Fat Free Mass | 188.0 lbs. | 70% |
| Fat | 81.0 lbs | 30% |
| ECT | 99.6 lbs. | 52% |
| Impedance Index | 1469 Normal | |

Time: 8:38 am Oct. 9, 1999

| | |
|---|---|
| Measured: Resistance: 442 ohms | Reactance: 56 ohms |
| Calculated: Impedance 445.5 ohms | Phase Angle: 7.2 degrees |
| Parallel Model: Resistance: 449.1 ohms | Capacitance: 898.0 pF |

| | | | | |
|---|---|---|---|---|
| Fluid Assessment: | | | | |
| Status: (Edema) | Results: | Percent: | Normal Range: | Deviation: |
| Total Body Water | 62.0 L | 51% (WT) | 40%-50% | +1 |
| Intracellular Water | 36.6 L | 60% (TBW) | 51%-60% | +0 |
| Extracellular Water | 25.4 L | 40% (TBW) | 39%-51% | +0 |

| | | |
|---|---|---|
| Nutrition Assessment: | | |
| Basal Metabolism | 2048 Kcal | |
| Body Cell Mass | 88.4 lbs. | 33% (WT) |
| Fat Free Mass | 187.5 lbs. | 70% |
| Fat | 81.5 lbs. | 30% |
| ECT | 99.1 lbs. | 53% |
| Impedance Index | 1426 Normal | |

Time: 8:43 am Oct. 9, 1999

| | |
|---|---|
| Measured: Resistance: 453 ohms | Reactance: 57 ohms |
| Calculated: Impedance 456.6 ohms | Phase Angle: 7.2 degrees |
| Parallel Model: Resistance: 460.2 ohms | Capacitance: 874.0 pF |

| | | | | |
|---|---|---|---|---|
| Fluid Assessment: | | | | |
| Status: (Edema) | Results: | Percent: | Normal Range: | Deviation: |
| Total Body Water | 63.6 L | 50% (WT) | 40%-50% | +0 |
| Intracellular Water | 36.2 L | 59% (TBW) | 51%-60% | +0 |
| Extracellular Water | 25.3 L | 41% (TBW) | 39%-51% | +0 |

| | | |
|---|---|---|
| Nutrition Assessment: | | |
| Basal Metabolism | 2040 Kcal | |
| Body Cell Mass | 87.6 lbs. | 33% (WT) |
| Fat Free Mass | 186.5 lbs. | 69% |
| Fat | 82.5 lbs. | 31% |
| ECT | 99.0 lbs. | 53% |
| Impedance Index | 1421 Normal | |

Time: 8:45 Consumed additional 500 ml Penta-hydrate Pro ™

Time: 8:48 a.m. Oct. 9, 1999

| | |
|---|---|
| Measured: Resistance: 431 ohms | Reactance: 60 ohms |
| Calculated: Impedance 435.2 ohms | Phase Angle: 7.9 degrees |
| Parallel Model: Resistance: 439.4 ohms | Capacitance: 1008.6 pF |

| | | | | |
|---|---|---|---|---|
| Fluid Assessment: | | | | |
| Status: (Edema) | Results: | Percent: | Normal Range: | Deviation: |
| Total Body Water | 62.5 L | 51% (WT) | 40%-50% | +1 |
| Intracellular Water | 37.9 L | 61% (TBW) | 51%-60% | +1 |
| Extracellular Water | 24.5 L | 39% (TBW) | 39%-51% | +0 |

| | | |
|---|---|---|
| Nutrition Assessment: | | |
| Basal Metabolism | 2078 Kcal | |
| Body Cell Mass | 91.7 lbs. | 34% (WT) |
| Fat Free Mass | 188.4 lbs. | 70% |
| Fat | 80.6 lbs. | 30% |
| ECT | 96.8 lbs. | 52% |
| Impedance Index | 1561 Normal | |

Time: 9:39 consumed 500 ml Penta-hydrate ™

Time: 9:07 am Oct. 9, 1999

| | |
|---|---|
| Measured: Resistance: 442 ohms | Reactance: 57 ohms |
| Calculated: Impedance: 445.7 ohms | Phase Angle: 7.3 degrees |
| Parallel Model: Resistance: 449.4 ohms | Capacitance: 913.5 pF |

| | | | | |
|---|---|---|---|---|
| Fluid Assessment: | | | | |
| Status: (Edema) | Results: | Percent: | Normal Range: | Deviation: |
| Total Body Water | 62.0 L | 51% (WT) | 40%-50% | +1 |
| Intracellular Water | 36.8 L | 59% (TBW) | 51%-60% | +0 |
| Extracellular Water | 25.2 L | 41% (TBW) | 39%-51% | +0 |

| | | |
|---|---|---|
| Nutrition Assessment: | | |
| Basal Metabolism | 2053 Kcal | |
| Body Cell Mass | 88.9 lbs. | 33% (WT) |
| Fat Free Mass | 187.5 lbs. | 70% |
| Fat | 81.5 lbs. | 30% |
| ECT | 98.6 lbs. | 53% |
| Impedance Index | 1452 Normal | |

Time: 9:27 am Oct. 9, 1999

| | |
|---|---|
| Measured: Resistance: 427 ohms | Reactance: 56 ohms |
| Calculated: Impedance 430.7 ohms | Phase Angle: 7.5 degrees |
| Parallel Model: Resistance: 434.3 ohms | Capacitance: 961.1 pF |

| | | | | |
|---|---|---|---|---|
| Fluid Assessment: | | | | |
| Status: (Edema) | Results: | Percent: | Normal Range: | Deviation: |
| Total Body Water | 62.7 L | 51% (WT) | 40%-50% | +1 |
| Intracellular Water | 37.4 L | 60% (TBW) | 51%-60% | +0 |
| Extracellular Water | 25.3 L | 40% (TBW) | 39%-51% | +0 |

| | | |
|---|---|---|
| Nutrition Assessment: | | |
| Basal Metabolism | 2066 Kcal | |
| Body Cell Mass | 90.3 lbs. | 34% (WT) |
| Fat Free Mass | 188.8 lbs. | 70% |
| Fat | 80.2 lbs. | 30% |
| ECT | 98.5 lbs. | 52% |
| Impedance Index | 1471 Normal | |

Time: 9:46 am Oct. 9, 1999

| | |
|---|---|
| Measured: Resistance: 430 ohms | Reactance: 59 ohms |
| Calculated: Impedance 434.0 ohms | Phase Angle: 7.8 degrees |
| Parallel Model: Resistance: 438.1 ohms | Capacitance: 996.9 pF |

| | | | | |
|---|---|---|---|---|
| Fluid Assessment: | | | | |
| Status: (Edema) | Results: | Percent: | Normal Range: | Deviation: |
| Total Body Water | 62.0 L | 51% (WT) | 40%-50% | +1 |
| Intracellular Water | 37.8 L | 60% (TBW) | 51%-60% | +0 |
| Extracellular Water | 24.7 L | 40% (TBW) | 39%-51% | +0 |

| | | |
|---|---|---|
| Nutrition Assessment: | | |
| Basal Metabolism | 2075 Kcal | |
| Body Cell Mass | 91.3 lbs. | 34% (WT) |
| Fat Free Mass | 188.5 lbs. | 70% |
| Fat | 80.5 bs. | 30% |
| ECT | 97.2 lbs. | 52% |
| Impedance Index | 1539 Normal | |

Time: 10:32 am Oct. 9, 1999

| | |
|---|---|
| Measured: Resistance: 437 ohms | Reactance: 57 ohms |
| Calculated: Impedance 440.7 ohms | Phase Angle: 7.4 degrees |
| Parallel Model: Resistance: 444.4 ohms | Capacitance: 934.2 pF |

| | | | | |
|---|---|---|---|---|
| Fluid Assessment: | | | | |
| Status: (Edema) | Results: | Percent: | Normal Range: | Deviation: |
| Total Body Water | 62.2 L | 51% (WT) | 40%-50% | +1 |
| Intracellular Water | 37.0 L | 60% (TBW) | 51%-60% | +0 |
| Extracellular Water | 25.2 L | 40% (TBW) | 39%-51% | +0 |

| | | |
|---|---|---|
| Nutrition Assessment: | | |
| Basal Metabolism | 2058 Kcal | |
| Body Cell Mass | 89.5 lbs. | 33% (WT) |
| Fat Free Mass | 187.9 lbs. | 70% |
| Fat | 81.1 lbs. | 30% |
| ECT | 98.4 lbs. | 52% |
| Impedance Index | 1466 Normal | |

Although test subjects were well hydrated prior to testing, the results were dramatic. Analysis of the above tests clearly show rapid cellular fluid exchange not possible with current hydrating fluid hydrating technology, including intravenous hydration methods. Similar tests utilizing tap and purified water demonstrated no change in cellular fluid exchanges over the same time frames. Note even though over-hydration increased total body water, the intercellular and extracellular remained within normal range with rapid noted in and out exchanges seen in both intercellular and extracellular fluids. And a 1.0% decrease in edema is noted after consuming only 500 ml of Penta-hydrate™ micro-cluster water. It is worth noting that the base micro-cluster water without oxygen is even more dramatic, hydrating the cells in less time than the oxygenated version micro-cluster water. The overall change in the Impedance Index of 124 points is utilized by the RJA System as an overall indication of health. Changes of this magnitude are not seen in a 90 day period of monitoring in the absence of oxygenated micro-cluster water (Pentahydrate™ Micro-Cluster Water). However, when Penta-hydrate™ Micro-Cluster Water was consumed the 124 point change occurred within a 2.5 hour period.

### EXAMPLE 3

A novel water prepared by the method of the invention was characterized with respect to various parameters.

A. Conductivity

Conductivity was tested using the USP 645 procedure that specifies conductivity measurements as criteria for characterizing water. In addition to defining the test protocol, USP 645 sets performance standards for the conductivity measurement system, as well as validation and calibration requirements for the meter and conductivity. Conductivity testing was performed by West Coast Analytical Service, Inc. in Santa Fe Springs, Calif.

Conductivity Test Results

| | | Micro-cluster | Micro-cluster |
|---|---|---|---|
| w/0₂ | RO Water | Water | Water |
| Conductivity at 25° C.* | 5.55 | 3.16 | 3.88 |
| (μmhos/cm) | | | |

*Conductivity values are the average of two measurements.

The conductivity observed for the micro-cluster water is reduced by slightly more than half compared to the RO water. This is highly significant and indicates that the micro-cluster water exhibits significantly different behavior and is therefore substantively different, relative to RO unprocessed water.

B. Fourier Transform Infra Red Spectroscopy (FTIR)

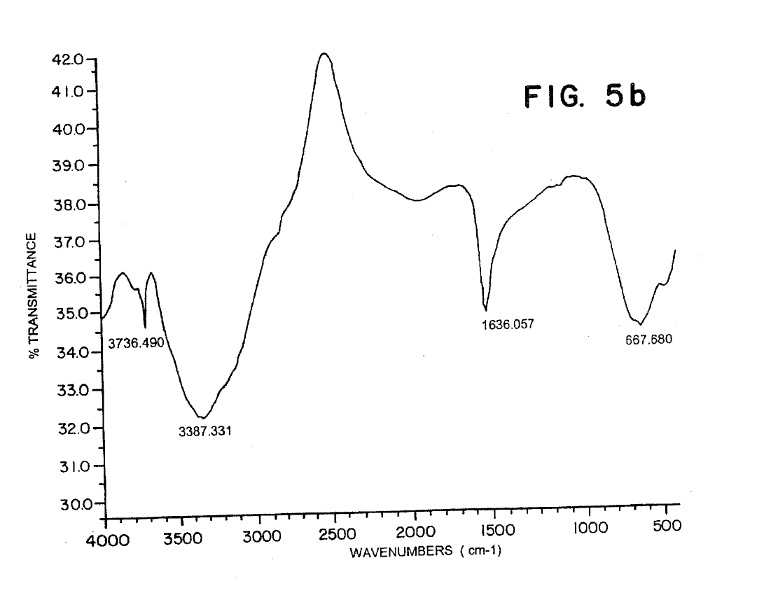

Water, a strong absorber in the IR spectral region, has been well-characterized by FTIR and shows a major spectral line at approximately 3000 wave numbers corresponding to O—H bond vibrations. This spectral line is characteristic of the hydrogen bonding structure in the sample. An unprocessed RO water sample, Sample A, and a unoxygenated micro-cluster water sample, Sample B, were each placed between silver chloride plates, and the film of each liquid analyzed by FTIR at 25° C. The FTIR tests were performed by West Coast Analytical Service, Inc. in Santa Fe Springs, Calif. using a Nicolet Impact 400D™ benchtop FTIR. The FTIR spectra are shown in FIG. 5.

In comparing the FTIR spectra for the unoxygenated micro-cluster and RO waters, it is clear that the two samples have a number of features in common, but also significant differences. A major sharp feature at approximately 2650 wave numbers in the FTIR spectrum is observed for the micro-cluster water (FIG. 5(b)). The RO water has no such feature (FIG. 5(a)). This indicates that the bonds in the water sample are behaving differently and that their energetic interaction has changed. These results suggest that the unoxygenated micro-cluster water is physically and chemically different than RO unprocessed water.

C. Simulated Distillation

Simulated distillations were carried out on RO water and unoxygenated micro-cluster water without oxygenation by West Coast Analytical Service, Inc. in Santa Fe Springs, Calif.

Simulated Distillation Test Results

| | RO Water | Unoxygenated Micro-cluster Water |
|---|---|---|
| Boiling Point range* | 98-100 | 93.2-100 |
| (deg. C.) | | |

*Corrected for barometric pressure.

These results show a significant lowering of the boiling temperature of the lowest boiling fraction in the unoxygenated micro-cluster water sample. The lowest boiling fraction for micro-cluster water is observed at 93.2° C. compared with a temperature of 98° C. for the lowest boiling fraction of RO water. This suggests that the process has significantly changed the compositional make-up of molecular species present in the sample. Note that lower boiling species are typically smaller, which is consistent with all observed data and the formation of micro-clusters.

D. Thermogravimetric Analysis

In this test, one drop of water was placed in a dsc sample pan and sealed with a cover in which a pin-hole was precision laser-drilled. The sample was subject to a temperature ramp increase of 5 degrees every 5 minutes until the final temperature. TGA profiles were run on both unoxygenated micro-cluster water and RO water for comparison.

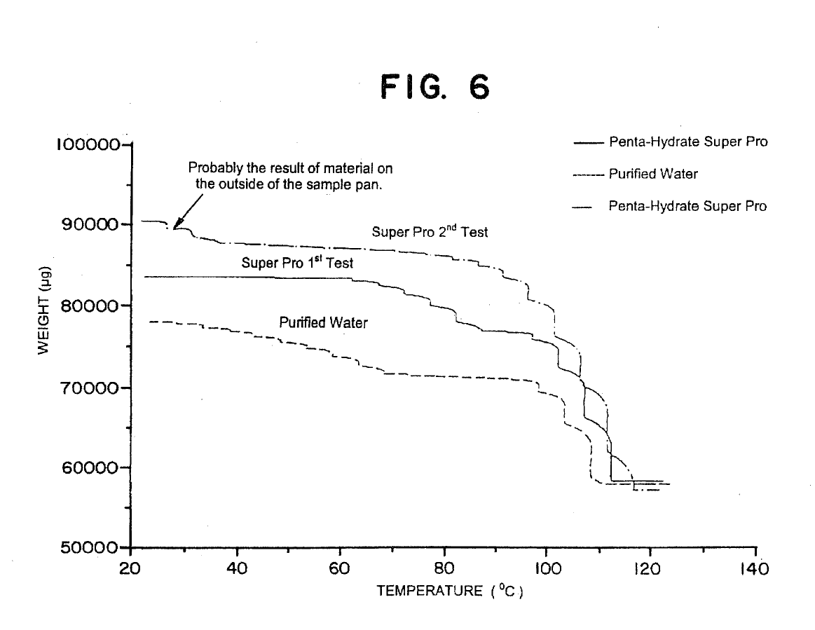

The TGA analysis was performed on a TA Instruments Model TFA2950™ by Analytical Products in La Canada, Calif. The TGA test results are shown in FIG. 6. Three test runs utilizing three different samples are shown. The RO water sample is designated, “Purified Water” on the TGA plot. The unoxygenated micro-cluster water was run in duplicate, designated Super Pro 1^{St} test and Super Pro 2^{nd} Test. The unoxygenated micro-cluster water and the unprocessed RO water showed significantly greater weight loss dynamics. It is evident that the RO water began losing mass almost immediately, beginning at about 40° C. until the end temperature. The micro-cluster water did not begin to lose mass until about 70° C. This suggests that the processed water has a greater vapor pressure between 40 and 70° C. compared to unprocessed RO water. The TGA results demonstrated that the vapor pressure of the unxoygenated micro-cluster water was lower when the boiling temperature was reached. These data once again show that the unoxygenated micro-cluster water is significantly changed compared to RO water. These data once again show that the unoxygenated micro-cluster water also shows more features between the temperatures of 75 and 100+deg. C. These features could account for the low boiling fraction(s) observed in the simulated distillation.

E. Nuclear Magnetic Resonance (NMR) Spectroscopy

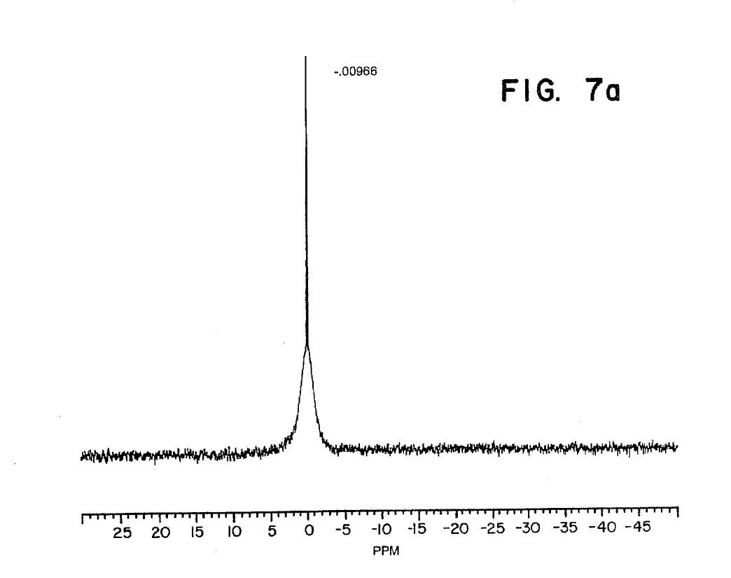

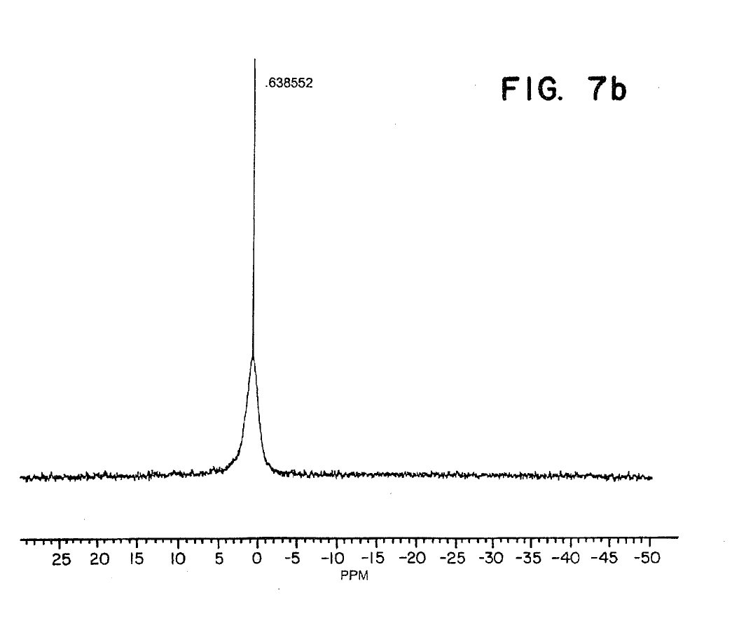

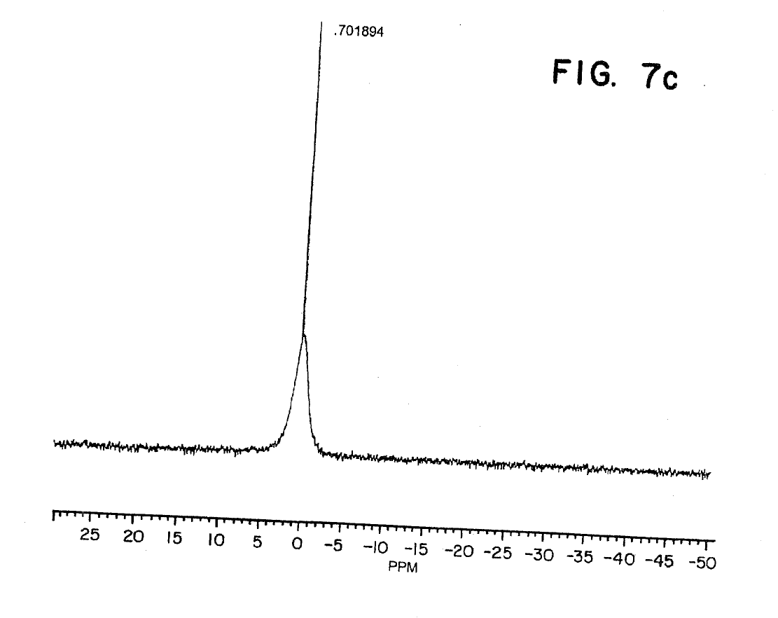

NMR testing was performed by Expert Chemical Analysis, Inc. in San Diego, Calif. utilizing a 600 MHz Bruker AM500™ instrument. NMR studies were performed on micro-cluster water with and without oxygen and on RO water. The results of these studies are shown in FIG. 7. In ¹⁷O NMR testing a single expected peak was observed for RO water (FIG. 7(a)). For micro-cluster water without oxygen (FIG. 7(b)), the single peak observed was shifted +54.1 Hertz relative to the RO water, and for the micro-cluster water with oxygen (FIG. 7(c)), the single peak was shifted +49.8 Hertz relative to the RO water. The shifts of the observed NMR peaks for the micro-cluster water and RO water. Also of significance in the NMR data is the broadening of the peak observed with the micro-cluster water sample compared to the narrower peak of the unprocessed sample.

### EXAMPLE 4

### Raman Spectroscopy

Raman spectroscopy, which is highly sensitive to structural modification of liquids, was employed to characterize and differentiate micro-cluster structures and micro-clustered molecular structure liquids. This study was based on obtaining and processing spontaneous Raman spectra and allowing a registration of types of phase transition in liquid water at 4, 19, 36 and 75 degrees Celsius. The hydrogen bond network and the average per unit volume hydrogen bond concentration were determined, which led to characterization of waters produced by different methods and in particular differentiation and definition of water composition produced by the methods described above for making micro-clusters.

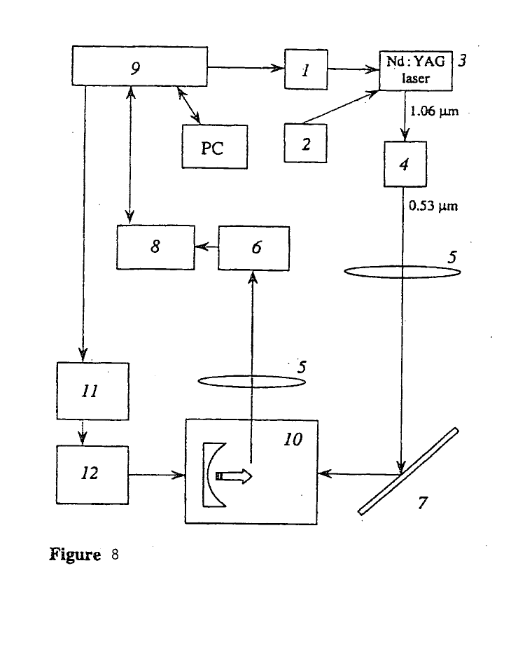

FIG. 8 schematically illustrates the device used in these studies. The source of illumination was a Q-switched solid state Nd:YAG laser (Spectra Physics Corp., Mountain View, Calif.) with two harmonics output at 1064 nm and its doubled frequency to produce a wavelength of 532 nm. A second harmonic generator comprised a KTP crystal available from Kigre, Tuscon, Ariz. The first harmonic was at 1064 nm with a pulse energy of 200 mJ, width of 10 ns, and repetition rate of 6 Hz. The optical mirror and translucent cell were obtained from CVC Optics, Albuquerque, N. Mex. The spectrometer was obtained from Hamamatsu (Japan), and its auto-collimation system from Newport Corporation, Costa Mesa, Calif. The electro-optical converter was from Texas Instruments, Houston, Tex.

The cell was filled with water as a test subject. The following water samples were studied: oxygenated micro-cluster water, unoxygenated micro-cluster water, Millipore (tm) distilled water, distilled water prepared in the laboratory, medical-grade double distilled injection water, bottled commercial reverse osmosis water, and tap water (unprocessed). The test water was subjected to strong ultrasonic fields produced by a pulse generator and a sine wave generator and a focusing horn. A laser beam was directed into a cell. Signals scattered at 90 degrees entered the spectrometer, which contained a grating unit providing a dispersion of 2 nm/mm. A Raman scattering spectrum was measured by a detector.

The results indicated the modifications in micro-cluster water of the local structure of the hydrogen-bond net in the acoustic field. In particular, the modification corresponded to a local decrease of the average distance between oxygen atoms to 2.80 angstroms, enhancing the ordering of the net structure of hydrogen-bonded water molecules to nearly that of hexagonal ice, where this distance is 2.76 angstroms.

The test samples which contained micro-cluster water were shown to have about a ten degree Celsius higher cluster temperature compared to the other water samples, which indicated that the average cluster size was smaller in the micro-cluster waters than in the other water samples. Further, the micro-cluster waters represented a more homogeneous composition of cluster sizes than the other waters, i.e. a more homogenous molecular cluster structure.

### Culture Media, Methods of Making and Using

The present invention involves compositions of culture media for biological, agricultural, pharmaceutical, industrial, and medical uses. The compositions comprise micro-cluster water. Methods of making and using the culture media compositions are within the scope of the invention.

General Description and Definitions

The practice of the present invention will employ, unless otherwise indicated, conventional techniques within the skill of the art in (1) culturing animal cells, plant cells, and tissues thereof; microorganisms, subcellular parts, viruses, and bacteriophage; (2) perfusion of differentiated tissues and organs; (3) biochemistry; (4) molecular biology; (5) microbiology; (6) genetics; (7) chemistry. Such techniques are explained fully in the literature. See, e.g. Culture of Animal Cells: A Manual of Basic Technique, 4th edition, 2000, R. Ian Freshney, Wiley Liss Publishing; Animal Cell Culture, eds. J. W. Pollard and John M. Walker; Plant tissue Culture: Theory and Practice, 1983, Elsevier Press; Plant Cell Culture Secondary Metabolism Toward Industrial Application, Frank DiCosmo and Masanaru Misawa, CRC Press; Plant Tissue Culture Concept and Laboratory Exercises, 2nd edition, Robert N. Trigiano and Dennis Gray, 1999, CRC Press; Plant Biochemistry and Molecular Biology, 2nd ed., eds. Peter J. Lea and Richard C. Leegood, 1999, John Wiley and Sons; Experiments in Plant Tissue Culture, Dodds & Roberts, 3rd edition; Neural Cell Culture: A Practical Approach, vol. 163, ed. James Cohen and Graham Wilkin; Maniatis et al., Molecular Cloning: A Laboratory Manual; Molecular Biology of The Cell, Bruce Alberts, et. al., 4th edition, 2002, Garland Science: Microbial Biotechnology, Fundamentals of Applied Microbiology, Alexander N. Glazer and Hiroshi Nikaido 1995, W. H. Freeman Co.; Pharmaceutical Biotechnology, eds. Daan J. A. Crommelin and Robert D. Sindelar, 1997, Harwood Academic Publishers). Relevant periodicals include Cell Tissue Research; Cell; Science; Nature; Journal of Immunology; Thymus; International Journal of Cell Cloning; Blood; Hybridoma.

The following terminology will be used in accordance with the definitions set out below in describing the present invention.

The term “micro-clustered culture medium” as used herein refers to a culture medium which comprises micro-cluster water. The adjective “micro-clustered” which modifies any of the aqueous compositions including medium, media, liquid, gel, composition, constituent or ingredient refers to micro-clustered water in that composition, i.e. which is dissolved in or mixed with micro-cluster water.

As defined in the Oxford Dictionary of Biochemistry and Molecular Biology (Oxford University Press, 1997), the term “culture” refers to 1(a) a collection of cells, tissue fragments, or an organ that is growing or being kept alive in or on a nutrient medium (i.e. culture medium); (b) any culture medium to which such living material has been added, whether or not it is still alive. 2. the practice or process of making, growing, or maintaining such a culture. 3. to grow, maintain or produce a culture.

A “cell” is the basic structural unit of all living organisms, and comprises a small, usually microscopic, discrete mass of organelle-containing cytoplasm bounded externally by a membrane and/or cell wall. Eukaryotes are cells which contain a cell nucleus enclosed in a nuclear membrane. Prokaryotes are cells in which the genomic DNA is not enclosed by a nuclear membrane within the cells.

“Culture medium” refers to any nutrient medium that is designed to support the growth or maintenance of a culture. Culture media are typically prepared artificially and designed for a specific type of cell, tissue, or organ. They usually consist of a soft gel (often referred to as solid or semi-solid medium) or a liquid, but occasionally they are rigid solids.

“Tissue culture” refers to 1. the technique or process of growing or maintaining tissue cells (cell culture), whole organs (organ culture) or parts of an organ, from an animal or plant, in artificial conditions; 2. any living material grown or maintained by such a technique.

“Tissue” refers to any collection of cells that is organized to perform one or more specific function.

“Organ” is any part of the body of a multicellular organism that is adapted and/or specialized for the performance of one or more vital functions.

“Organ culture” refers to a category of tissue culture, in which an organ or part of an organ, or an organ primordium, after removal from an animal or plant, is maintained in vitro in a nutrient medium with retention of its structure and/or function.

“Organelle” is any discrete structure in a unicellular organism or in an individual cell of a multicellular organism, that is adapted and/or specialized for the performance of one or more vital functions.

“Microbial biotechnology” refers to the use of cells, prokaryotic or eukaryotic, in production of proteins, recombinant and synthetic vaccines, microbial insecticides, enzymes, polysaccharides and polyesters, ethanol, amino acids, antibiotics; in organic synthesis and degradation by microbes (and by enzymes); and to environmental applications, including sewage and wastewater microbiology; microbial degradation of xenobiotics; use of microorganisms in mineral recovery, and in removal of heavy metals from aqueous effluents. The broad scope of microbial biotechnology is, in part, disclosed in Microbial Biotechnology, Fundamentals of Applied Microbiology, Alexander N. Glazer and Hiroshi Nikaido 1995, W. H. Freeman Co.; Pharmaceutical Biotechnology, eds. Daan J. A. Crommelin and Robert D. Sindelar, 1997, Harwood Academic Publishers.

Cell Culture Media—Fundamentals

The basic ingredients (as set forth below) of cell culture media—as individual components, as premixed components, dry or formulated with water—are commercially available from many vendors (e.g. Sigma Chemical, Invitrogen, Biomark, Cambrex, Clonetics to name just a few). Methods of formulating culture media with water are well known in the art (Culture Media for Cells, Organs, and Embryos, CRC Press, 1977; Animal Cells: Culture and Media: Essential Data, John Wiley & Son, 1995; Methods for Preparation of Media, Supplements and Substrata for Serum Free Animal Cell Culture in Cell Culture Methods for Molecular and Cell Biology, Vol. 1, Wiley-Liss, 1984). The media compositions of the invention comprise micro-cluster water. For the sake of listing the various ways of cell culturing Methods of cell culturing and types of cell media are well known in the art, and are briefly set forth below.

Types of Cell Cultures:

Primary cultures are taken directly from excised, normal animal tissue. These tissues are cultured either as an explant culture or cultured after dissociation into a single cell suspension by enzyme digestion. At first heterogeneous, these cultures are later dominated by fibroblasts. Generally, primary cultures are maintained in vitro for limited periods, during which primary cells usually retain many of the differentiated characteristics of the cells seen in vivo.

Continuous Cultures are comprised of a single cell type. These cells may be serially propagated in culture either for a limited number of cell divisions (approximately fifty) or otherwise indefinitely. Some degree of differentiation is maintained. Cell banks must be set up to maintain these cultures over long periods.

Culture Morphology

Cell cultures either growing in suspension (as single cells or small free-floating clumps) or as a monolayer attached to the tissue culture flask. Sometimes cell cultures may grow as semi-adherent cells in which there is a mixed population of attached and suspension cells.

Types of Culture Media

In general, cultured cells require a sterile environment, a supply of nutrients for growth, and a stable culture environment, e.g. pH and temperature. Various defined basal media types have been developed and are now available commercially. These have since been modified and enriched with amino acids, vitamins, fatty acids and lipids. Consequently media suitable for supporting the growth of a wide range of cell types are now available. The precise media formulations have often been derived by optimizing the concentrations of every constituent.

Vendors of culture media distribute via catalogs or the vendors' web sites to those skilled in the art literature for making and using culture media. For example, the Sigma-Aldrich company's web site discloses a book entitled Fundamental Techniques in Cell Culture, A Laboratory Handbook Online (Sigma-Aldrich Company), examples of different media and their uses are given in the table below. One of skill in the art would substitutes micro-clustered water for all or part of the non-micro-clustered water in the culture media recited below.

Table 1. Different Types of Culture Medium and their Uses

Balanced salt solutions PBS, Hanks BSS, Earles salts

DPBS (Prod. No. D8537/D8662)

HBSS (Prod. No. H9269/H9394)

EBSS (Prod. No. E2888) Form the basis of many complex media

Basal media MEM (Prod. No. M2279) Primary and diploid cultures.

DMEM (Prod. No. D5671) Modification of MEM containing increased level of amino acids and vitamins. Supports a wide range of cell types including hybridomas.

GMEM (Prod. No. G5154) Glasgows modified MEM was defined for BHK-21 cells Complex media RPMI 1640

(Prod. No. R0883) Originally derived for human leukaemic cells. It supports a wide range of mammalian cells including hybridomas

Iscoves DMEM

(Prod. No. 13390) Further enriched modification of DMEM which supports high density growth Leibovitz L-15

(Prod. No. L5520, liquid) Designed for CO2 free environments

TC 100 (Prod. No. T3160)

Grace's Insect Medium

(Prod. No. G8142)

Schneider's Insect Medium (Prod. No. S0146) Designed for culturing insect cells

Serum Free Media CHO (Prod. No. C5467)

HEK293 (Prod. No. G0791) For use in serum free applications.

Ham F 10 and derivatives

Ham F12 (Prod. No. N4888)

DMEM/F12 (Prod. No. D8062) NOTE: These media must be supplemented with other factors such as insulin, transferrin and epidermal growth factor. These media are usually HEPES buffered

Insect cells Sf-900 II SFM, SF Insect-Medium-2 (Prod. No. S3902) Specifically designed for use with Sf9 insect cells

Basic Ingredients of Media

Solutions of basic ingredients of media which comprise micro-clustered water are included in the compositions of the invention.

Inorganic salts

Carbohydrates

Amino Acids

Vitamins

Fatty acids and lipids

Proteins and peptides

Serum

Each type of constituent performs a specific function as outlined below:

Inorganic salts help to retain the osmotic balance of the cells and help regulate membrane potential by provision of sodium, potassium and calcium ions. All of these are required in the cell matrix for cell attachment and as enzyme cofactors.

Buffering Systems. Most cells require pH conditions in the range 7.2-7.4 and close control of pH is essential for optimum culture conditions. There are major variations to this optimum. Fibroblasts prefer a higher pH (7.4-7.7) whereas, continuous transformed cell lines require more acid conditions pH (7.0-7.4). Regulation of pH is particularly important immediately following cell seeding when a new culture is establishing and is usually achieved by one of two buffering systems; (i) a “natural” buffering system where gaseous CO2 balances with the CO3/HCO3 content of the culture medium and (ii) chemical buffering using a zwitterion called HEPES (Prod. No. H4034).

Cultures using natural bicarbonate/CO2 buffering systems need to be maintained in an atmosphere of 5-10% CO2 in air usually supplied in a CO2 incubator. Bicarbonate/CO2 is low cost, non-toxic and also provides other chemical benefits to the cells.

HEPES (Prod. No. H4034) has superior buffering capacity in the pH range 7.2-7.4 but is relatively expensive and can be toxic to some cell types at higher concentrations. HEPES (Prod. No. H4034) buffered cultures do not require a controlled gaseous atmosphere.

Most commercial culture media include phenol red (Prod. No. P3532/P0290) as a pH indicator so that the pH status of the medium is constantly indicated by the color. Usually the culture medium should be changed/replenished if the color turns yellow (acid) or purple (alkali).

Carbohydrates. The main source of energy is derived from carbohydrates generally in the form of sugars. The major sugars used are glucose and galactose however some media contain maltose or fructose. The concentration of sugar varies from basal media containing 1 g/l to 4.5 g/l in some more complex media. Media containing the higher concentration of sugars are able to support the growth of a wider range of cell types.

Vitamins. Serum is an important source of vitamins in cell culture. However, many media are also enriched with vitamins making them consistently more suitable for a wider range of cell lines. Vitamins are precursors for numerous co-factors. Many vitamins especially B group vitamins are necessary for cell growth and proliferation and for some lines the presence of B12 is essential. Some media also have increased levels of vitamins A and E. The vitamins commonly used in media include riboflavin, thiamine and biotin.

Proteins and Peptides. These are particularly important in serum free media. The most common proteins and peptides include albumin, transferrin, fibronectin and fetuin and are used to replace those normally present through the addition of serum to the medium.

Fatty Acids and Lipids. Like proteins and peptides these are important in serum free media since they are normally present in serum. e.g. cholesterol and steroids essential for specialized cells.

Trace Elements. These include trace elements such as zinc, copper, selenium and tricarboxylic acid intermediates. Selenium is a detoxifier and helps remove oxygen free radicals.

It is time consuming to make media from the basic ingredients, and there is a risk of contamination in the process. Conveniently, most media are available as ready mixed powders or as 10× and 1×liquid media. The commonly used media are listed in the catalogs of media vendors (e.g. Sigma-Aldrich Life Science Catalogue).

If one skilled in the art purchases media ingredients as powder or 10×media, it is essential that the water used to reconstitute the powder or dilute the concentrated liquid is free from mineral, organic and microbial contaminants. It must also be pyrogen free (Prod. No. W3500, water, tissue culture grade, Sigma-Aldrich). In most cases water prepared by reverse osmosis and resin cartridge purification with a final resistance of 16-18Mx is suitable. Once prepared the media should be filter sterilized before use. Obviously purchasing 1×liquid media direct from a vendor eliminates the need for this. In all instances, media of the invention involve micro-clustered water, preferably tissue culture grade, as a constituent. Vendors of media (e.g. Sigma-Aldrich, Invitrogen, Clonetics) and vendors of cells and cell cultures commonly purvey one or more of their products (media, media ingredients, and cells) in the form of kits which have containers for the products. The invention includes kits which comprise micro-clustered in its own container or as an ingredient of another container in the kit.

Serum. Serum is a complex mix of albumins, growth factors and growth inhibitors and is probably one of the most important components of cell culture medium. The most commonly used serum is fetal bovine serum. Other types of serum are available including newborn calf serum and horse serum. The quality, type and concentration of serum can all affect the growth of cells and it is therefore important to screen batches of serum for their ability to support the growth of cells. Serum is also able to increase the buffering capacity of cultures that can be important for slow growing cells or where the seeding density is low (e.g. cell cloning experiments).

The culture media of the invention, which comprise micro-clustered water, and methods of making and using them are arbitrarily classified for purposes of this application into use for the following categories of biological entities. It is understood that this classification does not preclude the compositions or their methods of use from application in more than one category.

Animal Cell, per se (e.g., Cell Lines, etc.)

Compositions of the invention include:

1. A composition comprising micro-clustered culture medium, in particular medium formulated for use with animal cells.

2. A composition comprising micro-clustered culture medium formulated for use with animal cells, and animal cells.

3. Compositions comprising animal cells made from using micro-clustered animal cell culture media in methods enumerated below.

The culture media of the invention formulated for use with animal cells are used for:

1. Propagating, maintaining or preserving an animal cell or composition thereof.

2. Isolating or separating an animal cell or composition thereof.

3. Preparing a composition containing an animal cell.

Also covered by the invention are processes for preparing micro-clustered animal cell culture media, and for preparing compositions which comprise micro-clustered animal cell culture medium and animal cells. Vaccines are examples of products derived from such animal cell cultures.

Stem Cells

The compositions and methods of the invention are adapted for use with stem cells. Embryonal stem cells and lineage- or tissue-specific stem cells are important models in biomedical studies, but the availability and accessibility of research materials in this rapidly advancing field often become limiting. The compounds and methods of the invention are intended for expanding, preserving embryonic stem cells, as well as postnatally derived stem cells from a variety of strains and species. (National Center for Research Resources; American Type Culture Collection, Manasas, Va.). Stem cells are also retrieved from bone marrow, subcutaneous fat, and the reticular dermis bulge area. Products available from the National Stem Cell Resource include: (a) nonhuman embryonic stem cells, and lineage- or tissue-specific neonatally derived stem cells from a variety of species; these are available as either frozen vials, shipped on dry ice; (b) selected reagents related to stem cell characterization and utilization are available; these include antibodies, nucleic acid probes, cDNAs, genomic libraries and plasmid vectors for targeted mutagenesis or other stem cell-related purposes; (c) standardized media, as they are developed. Reagents identifying common traits among stem cell strains and species also will be available as they are identified or developed. These include reagents for RT-PCR and immunologically based assays. The present invention includes use of micro-clustered media and reagents for use with stem cells, including stem cell retrieval.

Microorganisms

Microorganisms include actinomycetales, unicellular algae, bacteria, fungi (yeast and molds), and protozoa.

Compositions of the invention include

1. Culture media comprising micro-cluster water for use with microorganisms.

2. Culture media comprising micro-cluster water and microorganisms.

The culture media of the invention involved with microorganisms are used for:

1. Propagating, maintaining or preserving microorganisms, or compositions of microorganisms.

2. Preparing or isolating a composition containing a microorganism, which processes involve the use of micro-cluster water or culture media comprising micro-cluster water.

3. Isolating microorganisms.

Also covered by the invention are processes for preparing culture media comprising micro-cluster water, and for preparing compositions which comprises culture media and microorganisms.

Vector, per se (e.g., Plasmid, Hybrid Plasmid, Cosmid, Viral Vector, Bacteriophage Vector, etc.)

These biological entities include self-replicating nucleic acid molecules which may be employed to introduce a nucleic acid sequence or gene into a cell; such nucleic acid molecules are designated as vectors and may be in the form of a plasmid, hybrid plasmid, cosmid, viral vector, bacteriophage vector, etc.

Vectors or vehicles may be used in the transformation or transfection of a cell. Transformation is the acquisition of new genetic material by incorporation of exogenous DNA. Transfection is the transfer of genetic information to a cell using isolated DNA or RNA cA plasmid is an autonomously replicating circular extrachromosomal DNA element. A hybrid plasmid is a plasmid which has been broken open, has had DNA from another organism spliced into it, and has been resealed. A cosmid is a plasmid into which phage lambda “cos” sites have been inserted

A viral vector (e.g., SV40, etc.) is a plant or animal virus which is specifically used to introduce exogenous DNA into host cells. A bacteriophage vector (e.g., phage lambda, etc.) is a bacterial virus which is specifically used to introduce exogenous DNA into host cells.

Virus or Bacteriophage

These biological entities include a virus or bacteriophage which is a microorganism that (a) consists of a protein shell around a nucleic acid core of either ribonucleic acid or deoxyribonucleic acid, and (b) is capable of independently entering a host microorganism, and (c) requires a host microorganism, having both ribonucleic acid and deoxyribonucleic acid to replicate.

Compositions of the invention include

1. A composition of micro-clustered medium formulated for use with virus or bacteriophage.

2. A composition of micro-clustered medium formulated for use with virus or bacteriophage, which composition comprises virus or bacteriophage.

The culture media of the invention involved with virus or bacteriophage are used for:

1. Preparing or propagating virus or bacteriophage.

2. Purifying virus or bacteriophage.

3. Producing viral subunits.

Propagation is limited to processes concerned with the multiplication of viruses and not with processes concerned with the artificial alteration of genetic material involving changes in the genotype of the virus. Such processes of artificial alteration of genetic material are intended for processes of mutation, cell fusion, or genetic modification, and include (1) producing a mutation in an animal cell, plant cell or microorganism, (2) fusing animal, plant, or microbial cells, (3) producing a stable and heritable change in the genotype of an animal cell, plant cell, or a microorganism by artificially inducing a structural change in a gene or by incorporation of genetic material from an outside source, or (4) producing a transient change in the genotype of an animal cell, plant cell, or microorganism by the incorporation of genetic material from an outside source.

A mutation is a change produced in cellular DNA which can be either spontaneous, caused by an environmental factor or errors in DNA replication, or induced by physical or chemical conditions. The processes of mutation included are processes directed to production of either directed or essentially random changes to the DNA of an animal cell, plant cell, or a microorganism without incorporation of exogenous DNA. It should be noted that in the art that incorporation of exogenous genetic material into a cell or microorganism or rearrangement of genetic material within a cell or microorganism is not necessarily considered a mutation.

In vitro mutagenesis, which is a method where cloned DNA is modified outside of the cell or microorganism and then incorporated into a cell or microorganism is not considered to be a mutation. Genetic material from an outside source may include chemically synthesized or modified genes. Transient changes effected by incorporation of genetic material from an outside source involve expression of one or more phenotypic traits encoded by said genetic material. A transient change is one which is passing or of short duration. Methods producing nongenetically encoded changes effected by a nucleic acid molecule, such as antisense nucleic acid are not considered mutations.

These compositions and processes involve use with viruses of all types, i.e., animal, plant, etc.

Plant Cell or Cell Line, per se (e.g. Transgenic, Mutant, etc.)

These biological entities include plant cells or cell lines, per se which may be transgenic, mutant, or products of other processes for obtaining plant cells.

The compositions of the invention include:

1. A composition comprising micro-clustered water and medium formulated for plant cells or cell lines.

2. A composition comprising micro-clustered water and medium formulated for plant cells or cell lines and plant cells.

The culture media of the invention involved with plant cells or cell lines are used for:

1. In-vitro propagating

2. Maintaining or preserving plant cells or cell lines.

3. Isolating or separating plant cells.

4. Regenerating plant cells into tissues, plant parts, or plants, per se, with or without genotypic change occurring. (Total Lab Systems, Ltd., New Zealand; e.g. Commercial Propagation of Orchids in Tissue Culture: Seed Flasking Methods. Orchid Manual Basics, Kay S. Greisen, 2000, American Orchid Society; Plant Tissue Culture Protocols as disclosed in Sigma-Aldrich Co. web site and catalogs)

Subccllular Parts

It is understood that the compositions of the invention include media formulated for subcellular parts of microorganisms, animal cells and plants, such as organelles, i.e. mitochondria, microsomes, chloroplasts, etc. These media are used for isolating or treating subcelular parts. Methods of making these media are included in the invention.

Media for Use With Differentiated Tissues or Organs

The invention includes micro-clustered media adapted for use with differentiated tissues or organs, including blood. These media are used for the maintenance of a differentiated tissue or organ, i.e. maintained in a viable state in a nutrient or life sustaining media.

Maintenance includes keeping an organ under conditions in which it produces a product (e.g., hormone) which is later recovered, or exhibits an activity (e.g. synthesis of a hormone).

Accordingly, the invention includes perfusion media formulated with micro-clustered water, which are used in processes for the maintenance of differentiated tissue or organs by continuously perfusing with a fluid, or compositions of the invention. U.S. Pat. Nos. 4,879,283; 4,873,230; and 4,798,824 (herein incorporated by reference) disclose solutions for perfusing and maintaining organs. D'Alessandro A M, Kalayoglu M, Sollinger H W, Pirsch J D, Southard J H, Belzer F O. Current status of organ preservation with University of Wisconsin solution. Arch Pathol Lab Med. 1991; 115(3):306-310; Viaspan (r), an organ perfusion and maintenance solution, manufactured by Barr Laboratories, Inc. and used for transplantation and viability preservation of organs and tissues.

Compositions of the invention include those formulated for freezing of differentiated tissues or organs, and used in processes for maintaining differentiated tissues or organs by freezing.

Compositions of the invention include those formulated for maintaining blood or sperm in a physiologically active state, and those formulated for methods of in vitro blood cell separation or treatment. Also included are compositions for artificial insemination.

It is understood that micro-clustered compositions of the invention include physiological solutions or aqueous media which may not contain nutrient ingredients yet still formulated having pH, buffer capacity, osmolarity, conductance, sterility and which otherwise are used alone or in combination with other physiological solutions to maintain living cells, tissues, organs, and organisms. Examples of physiological solutions include, but are not limited to, Ringer's solutions, saline solutions, buffer solutions. These solutions are commonly known and used in handling biological materials, and are apparent to those of ordinary skill in the art.

Stimulation of Growth or Activity Using Micro-Clustered Medium Effects of Micro-Cluster Water on Cellular Viability

A study was performed to determine the influence of micro-cluster water on cell viability as measured by cell membrane integrity.

A population of macrophages was subjected to growth medium which was formulated with micro-cluster water, and growth medium formulated with double distillated water (DDW).

Macrophages were obtained by mice. 2 ml of Hanks solution (10 mM HEPES, pH 7.2) was injected into the peritoneum of sacrificed mice. The solution, containing macrophages, was collected. The cell concentration was adjusted to 106 cells/ml with Hanks balanced salt solution.

Generally, 20 microliter aliquots of the cell suspension were placed on glass cover slips, incubated for 45 minutes in a wet chamber, and then washed with Hanks solution to remove the cells attached to the glass surface.

The integrity of the cell membranes was determined by double staining the cells with ethidium bromide (EthBr, Sigma) and fluoresceindiacetate (FDA, Sigma). A staining solution was used which contained 5 micrograms/ml of EthBr and 5 micrograms/ml of FDA. Cells with damaged cell membranes were counted. The method is based on the ability of EthBr to enter cells which have damaged membranes. The EthBr binds to DNA. EthBr has a bright red fluorescence when bound to DNA. FDA easily penetrates cells from the medium and is structurally transformed to fluorescein which has bright green fluorescence. Accordingly, cells with intact plasma membranes accumulate fluorescein, whereas cells with damaged cell membranes allows fluorescein to easily leave the cells. As a result of this double staining, after five minutes, one observed cells with intact plasma membranes which had green fluorescence. Cells which had damaged plasma membranes had red fluorescence.

In a first series of experiments, macrophages were incubated for 15 minutes in media containing EthBr and FDA. They were then thoroughly washed to remove free dyes in the extracellular media. Growth media was then replaced with 199 medium (199 Powder medium—Russia, Paneko) prepared with either DDW or with micro-cluster water. Dead cells were then counted.

In a second series of experiments, cells were incubated for 230 minutes in either 199 cell medium prepared with DDW or micro-cluster water. Cells were then appropriately stained to determine how many cells had died.

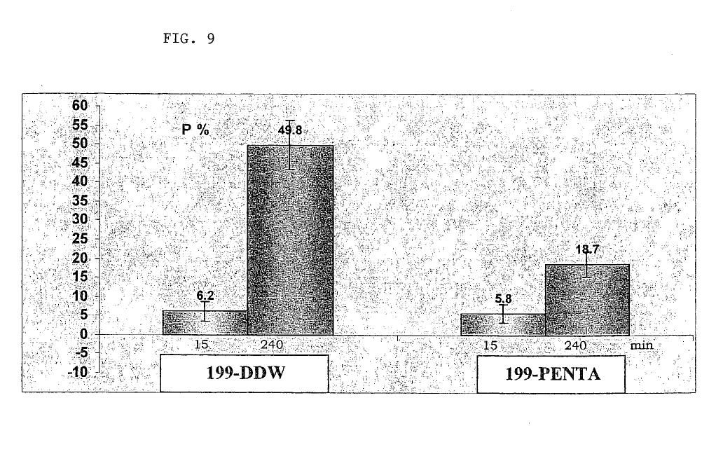

FIG. 9 is an assessment of the number of macrophages with damaged plasma membranes after incubation in 199 cel medium prepared on DDW or on micro-cluster water. The data is presented as percentage of cells with damaged plasma membranes—P %—after 15 minutes and 240 minutes of incubation in different 199 cell media. The results indicate that the amount of cells with damaged cell membranes was 2.6 times greater in cell medium prepared with double distilled water compared to medium prepared with micro-cluster water. Accordingly, it appeared that cell culture medium formulated with micro-cluster water prolonged or increased the life of cells compared with the effects of cell culture medium formulated with DDW. Alternatively, it appeared that cell culture medium prepared with micro-cluster water inhibited damage to cell plasma membranes compared to cell culture medium prepared with DDW.

Effects of Micro-cluster Water on Intracellular pH

A study was performed to determine the influence of micro-cluster water on intracellular pH.

Mouse macrophages were obtained as described above. Intracellular pH of these cells was determined after 15 minutes and after 240 minutes of incubation in 199 medium prepared either with DDW or micro-cluster water.

Macrophage intracellular pH was measured based on a microspectrophotometric method using a fluorescent microscope (LUMAM 13, LOMO, Russia), which as a modified system of fluorescence excitation and emission.

Fluorescence excitation was performed using a blue (lambda max=435 nm photodiode. Fluorescence was measured simultaneously at two different wavelengths by a two-channel system, which has lambdal=520 nm, lambda2=567 nm interference filters respectively.

Fluorescence excitation and synchronous emission measurement was achieved with a built-in microcontroller (LA-70M4).

Macrophages were incubated with fluorescent FDA (5 micrograms/ml), which is a pH indicator, for 15 minutes. After incubation with the dye, the cells were washed free from dye in the surrounding medium. The cells were then placed in the medium in a small petri dish, and observed using a water immersion objective (x40). A pH calibration curve was established for a range of ionic conditions.

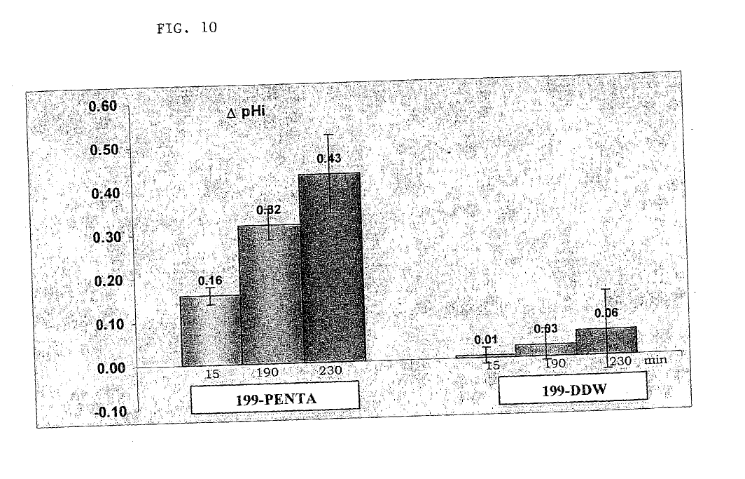

Cells, which had been incubated with FDA dye for 15 minutes and washed free from dye in the surrounding medium, were then placed in either 199 medium prepared with DDW or with micro-cluster water. Kinetic measurements of intracellular pH were made with no less than 30 microscopic observations, and repeated three times. Cells were incubated for as 1 cng as 230 minutes. FIG. 10 illustrates the kinetics of intracellular pH change (delta pHi) in macrophages after replacement of incubation medium with 199 medium prepared either with DDW or with micro-cluster water. The x-axis is time in seconds after change of cell medium. The y-axis is changes in intracellular pH—delta pHi. It can be seen that the intracelluar pH in a standard incubation medium 199-DDW and in 199-micro-cluster water were both about pH 7.15. After 15 minutes of incubation in 199-micro-cluster water, the pH increased by 0.16 unites. No significant change was observed in macrophages incubating in 199-DDW during the same 15 minutes. After 230 minutes, a 0.43 increase was observed in the intracellular pH of the cells incubating on 199-micro-cluster water. There was a negligible increase in intracellular pH of the cells incubating on 199-DDW. It is concluded that contacting cells with culture medium prepared with micro-cluster water instead of “normal” water increased the intracellular pH of the cells.

A separate series of experiments using pig embryo kidney cells cultured with 199 mediums and with 10% bovine serum demonstrated increases in intracellular pH and robust cell viability when the growth mediums were prepared with micro-clustered water compared to growth mediums prepared with normal water.

Effects of Micro-Cluster Water on Growth and Transfection of Two Types of Human Cells

A series of experiments was performed to determine the effects of micro-cluster water on the growth of cells and on the transfection of cells in medium prepared with micro-cluster water.

The effects were studied using human epithelial cells (293T) and human dendritic cells. DMEM medium (Life Technologies, Gaithersburg, Md.) was prepared from a 10×concentrate by dilution in micro-cluster water obtained from AquaPhotonics, Inc., San Diego, Calif.). The cells were supplemented with 10% fetal calf serum (FCS).

In a parallel experiment, the cells were cultured with standard DMEM medium, i.e. medium prepared without micro-clustered water.

At days 0, 3, 6, and 9 the cells were stained with 0.4% trypan blue (Life Technologies) to determine the viability of the culture.

On day 1 of culturing, the 293T cells were subjected to transfection with an HIV molecular clone (which encodes GFP) by a calcium phosphate precipitation method (Invitrogen, Carlsbad, Calif.). As a control, 293T cells cultured in standard DMEM medium were transfected with the same HIV molecular clone. The following day, supernatants were harvested from both HIV transfected cultures and assayed for HIV Gag p24 content by ELISA. To find optimal dilution in the range of sensitivity of the method, supernatants were titrated by a factor of 10.

The harvested viruses were then used to infect primary cultures of dendritic cells (DC). Two cultures of DC were maintained in the medium prepared from concentrated DMEM and diluted by a factor of 10, one culture (experimental) in DMEM diluted with micro-cluster water, the other culture (control) diluted with normal water. Infection was monitored at the single-cell level by scoring the GFP-positive DC at fifth day after HIV exposure.

Results:

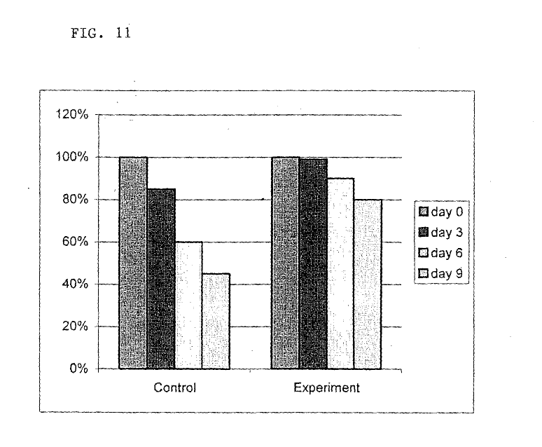

A. Viability tests, as shown in FIG. 11, demonstrated that the micro-cluster water used as a solvent for medium preparation, improved 293T cell viability by 70% at the 9th day of culture over the cells cultured in medium prepared with normal water.

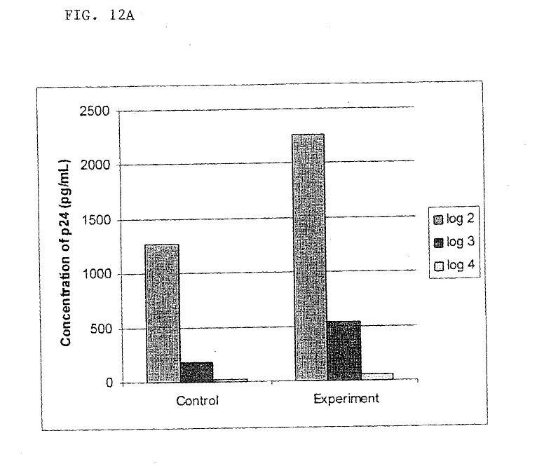

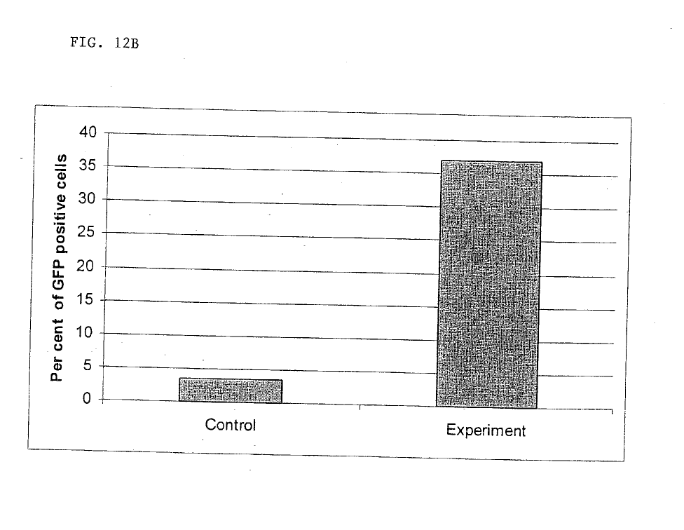

B. Replication of HIV in transfected 293T cell-cultures three-fold higher in the experimental cultures compared with the control cultures when supernatants from the respective cultures were titrated at the point of 3 log (FIG. 12a).

C. Culturing of DC in a DMEM medium prepared with micro-cluster water and exposure of DC to HIV harvested from 293T cells cultured in DMEM prepared with micro-cluster water greatly enhanced the “permissivity” (FIG. 12b) of DC to HIV (35% DC were infected in the experimental culture compared with 3.7% in the control.)

These experiments demonstrated in a transformed cell-line, in a virus, and in primary cells, biological effects on these biological entities when micro-clustered water replaced normal water in the culture medium. There was 2-3 fold enhancement of the cells' viability; and an augmentation of either or both HIV replication and replication rate in vitro in the cell line and in the primary cell culture.

Effects Of Micro-Cluster Water On The Expression Profiles of Characteristic Dendritic Cell Markers

This study's objective was to monitor difference between the expression profiles of characteristic DC markers in media prepared with de-ionized water and media prepared with micro-clustered water.

Experimental design and results. DC were cultured in media prepared from 10×-concentrate MEM (Life-Technologies, Gaitersburg, Md.) diluted to a final concentration either by de-ionized water or micro-clustered water. Both media were supplemented with cytokines IL-4 and GM-CSF (20 ng/ml). DC were generated according to standard protocols (Sallusto et al., 1994), phenotyped on day 6 of differentiation and cultured. On days 30 and 69, respectively, phenotyping was repeated with the same monoclonal antibodies. The level of surface-marker expression was assessed by flow cytometry using FACscan reader (Bekton-Dickenson, Calif.).

### Description of Cell Surface Markers

1. DC-SIGN—M.W. ˜44K, cell-specific ICAM-3 receptor Paper was attached about a function of DC-SIGN in dendritic cells.

2. CD4—main HIV gp120 receptor, MW ˜55K. CD4 is an anchor place for HIV envelope proteins

3. CD1a —is an analog of MHC complex in professional antigen-presenting cells, which is responsible for presentation and processing of lipid antigens (non canonical antigen-presentation system).

4. CD80—co-stimulatory molecule which provides signal 2 from antigen presenting cell (such as DC) for induction of T-cell proliferation.

5. CD83—maturation marker of dendritic cells (DC)

6. CXCR4 and CCR5—inflammatory chemokine receptors

7. MHC-II—Major Histo-Compatibility complex type II. Presents epitopes of exogeneous processed proteins.

As shown in FIG. 12, during long-term culturing in medium prepared with micro-clustered water as a solvent, it was observed that a substantial change occurred in the pattern of expression of the CD83 marker. CD83 is a main indicator of DC maturation. DCs that express a low level of CD83 on day 60 and show typical morphology (grown in suspension) are immature and functionally ready to take up foreign antigens. Typically, DCs exhibit such a phenotype in vitro (in standard medium) during first two weeks of differentiation. Further culturing in standard medium leads to a spontaneous maturation and cell-death mediated, most likely, through apoptosis. In a pilot phenotyping experiment it was detected that micro-clustered water (i) preserved immature DC phenotype and (ii) mediated DC surviving longer than 2.5 months. Phenotype preservation was shown by analysis of expression of other markers (most important are DC-SIGN and MHC II) on the surface of DC cells. This analysis indicates that micro-clustered medium provides a satisfactory maintenance of functions typical to immature DC as seen by similarity of markers expression between DC in standard and micro-clustered media.

The survival of DC's for more than 2.5 months was never observed before with standard medium formulation. Preliminary results demonstrated that micro-clustered water exhibited a biological activity reflected in modulation of DC cell surface markers.

Summary

1. Micro-clustered water was fully applicable as a solvent for fine tissue culture experiments.

2. Contacting the cells with micro-clustered water altered the cells' biological activity, which was reflected in modulation of CD83 marker and elongation of a lifetime span of DCs in vitro.

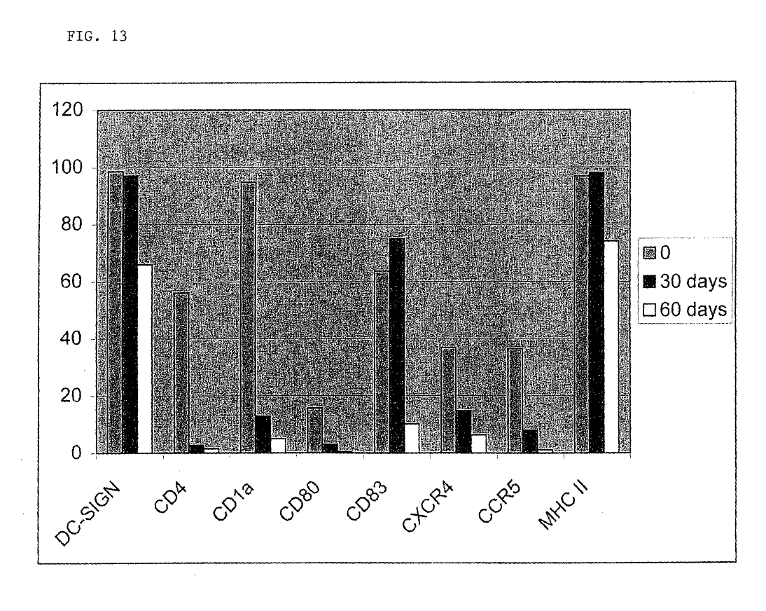

In FIG. 13, the horizontal axis reflects the type of different receptors on the cell surface. The vertical axis represents responses (percent of fluorescent intensity of labeled monoclonal antibodies bound to a specific receptors). Cells were stained with the respective monoclonal antibodies and signal was compared to the isotype control (percent of ˜ISO 1.1%).

In FIG. 13, the gray columns represent measurements after 6 days in control medium. The black columns after 30 days in control medium. The white columns after 60 days in micro-clustered medium. Data were not obtained for normal water in a day 60 since the cell culture underwent apoptosis at early date. Contrary to almost complete die-off of the cell in a standard medium, a surprisingly large number of cells in micro-structured medium showed a morphology of immature DC and a corresponding pattern of cell surface markers at day 60. Cell life survivability appeared to be enhanced by micro-clustered medium.

Effect of Micro-Clustered Water on the Functional State of Brain Tissue Perfused in Artificial Cerebrospinal Fluid Prepared with Double Distilled and Micro-Clustered Waters.

The purpose of the study was to measure the effect of various types of waters on the functional state of brain tissue. Recording of an induced electrical signal from the brain sections in perfused fluid due to activity of hippocampus nervous cells was used as the testing method. According to the literature, the technology of making rat brain sections with a hippocampus of 300-450 μm in perfusion with artificial cerebrospinal fluid allows the brain tissue to keep its functional status for about 6-8 hours.

The method employed involved testing of the functional status of brain tissue by recording electrical neuron responses to the applied pulses of electric current. Neuronal reaction is very sensitive to the characteristics of perfusion medium. Stimulation of the axon group reflects the change in membrane potential of postsynaptic cells, which are located in a region of the measuring electrode. The amplitude of the signal depends on the efficacy of the synaptic connections between stimulating axons and postsynaptic neurons and it also depends on the excitability of the postsynaptic neurons themselves. Declining functional activity of brain tissue is a result of a reduction in the neurons which are responding to the applied pulses of electric current. This is directly correlated with a decrease in summary amplitude.

The main advantages of the method involved easy access to the extracellular space of brain tissue in the specimen which made it possible to use chemical substances of required concentration directly. Furthermoe, there was an absence of interference due to respiration, heart beating, and animal movement, which make prolonged measurements difficult; experimental condition were easy to control in the absence of anesthesia, humoral, and hormonal influences. Also, it was relatively simple to use the tested tissue for biochemical and morphological analysis quickly after the electrophysiological part was completed.

Requirements for the survivability of brain tissue sections. To maintain viability of isolated brain sections, artificial fluids are used which are similar in salt composition to the intercellular medium of the brain. However, the composition of cerebrospinal fluid may vary depending on the specific task.

Glucose was used as an energetic substrate in fluid. The pH of the fluid was controlled with a bicarbonate buffer. Osmotic pressure was in the range of 294-311 mosm/l. The solution was also oxygenated by carbogen gas (mixture consisting of 95% oxygen and 5% CO2). Temperature was maintained in the range of 22-33 Celsius. Since the sections were without normal capillary blood flow, the exchange of substances was sustained due to the diffusion of oxygen, substrates, and metabolites between the incubation medium and the whole tissue section. Therefore, the thickness of the section had to be small enough to allow complete diffusion through the specimen. According to the empirical formula used in calculating the section thickness, the maximum value is approximately 600 microns and this depends on the intensity of the oxidation process. During isolation, section cells in the surface layers with a size of 100 microns are damaged. Pyramidal cells are approximately the same size, so the section depth should be at least 300 microns Experiments were conducted on brain tissue sections of Wistar rats, 1 month of age. Anesthesia was performed using ether. Rat brain was isolated and placed into cold artificial cerebrospinal (AC) fluid prepared with double distilled water. AC fluid composition: (mM): NaCl-130, KCl-3.5, NaH2*PO4-1.2, MgCl2-1.3, CaCl2-2.0, NaHCO3-25.0, and glucose. A Carbogen gas mixture was continuously pumped through the solution. Hippocampus sections of 400 microns were obtained using a vibratome.

The sections were then placed into an incubation chamber, which contained AC fluid, and maintained at 22-25 Celsius. After 1 hour in the incubation chamber, the sections were transferred separately to the testing chamber, with AC fluid flowing through it at the rate of 3-5 ml/min. Stimulating electrical pulses (100 msec, 100-400 mA) were been delivered through bipolar wolfram electrodes (200 mm), which were located on the Shaffer's collators (nerve fibers, ending exciting synapses in the CA1 region of hippocampus). Induced potentials, which are an electrical response to the stimulation of assembly/totality, were recorded in the CA1 region of hippocampus by using a glass microelectrode filled with AC fluid (resistance 0.5-1.0 mW).

Two series of experiments were performed. Standard AC fluid (A) was used as the initial 100% level of signal in both series. In the first series of experiments, AC fluid was replaced with the solution having the same salt composition and double distilled water—(solution B). In the second series of experiments, micro-clustered water replaced double distilled water in solution B (solution C).

The perfusion system utilized made it possible to continuously switch the supply of the solutions into the testing chamber. The complete substitution of one solution by another in the chamber with a volume of 2 ml occurred during 1 minute. The amplitude of induced response was the comparative characteristic. To measure the induced negative monophase response, which is 30-40% of the maximum amplitude for the parameters of power, duration and location of stimulation were selected. Testing was produced with a series of 10 single pulses with intervals of 10 msec. A series of pulses were applied at intervals of 2 to 10 minutes. The recorded signal was digitized by an analog-digital converter and was saved for the following analysis. Final data processing was completed using Excel and Origin software. Statistical analysis was performed using paired t tests. The value of P<0.05 was accepted as being statistically significant.

Results. Shaffer's collators were stimulated in the CA1 region and the induced response was recorded after 0.5-4 msec and from 4-6 msec.

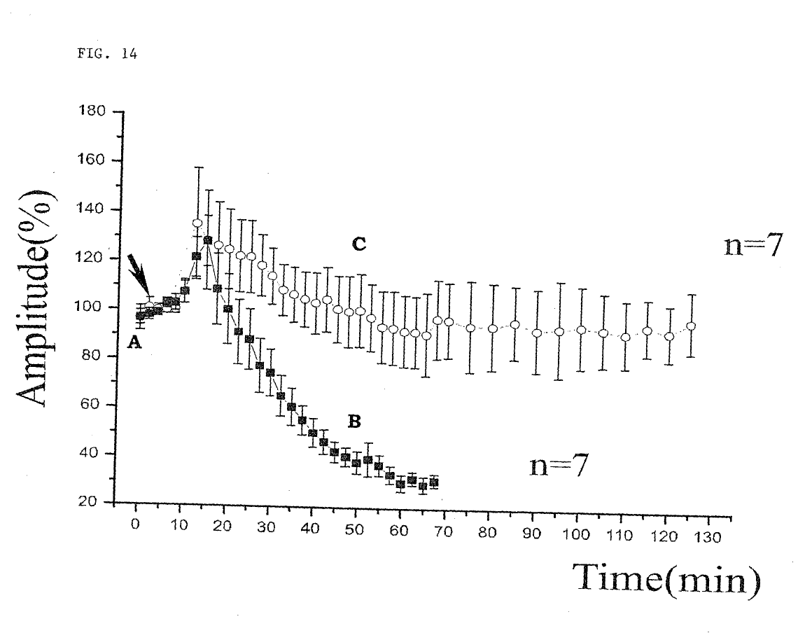

FIG. 14 shows the dependence of focal potential measured from the rat hippocampus on the type of perfusing fluid. The horizontal axis represents the time after the beginning of the experiment. The vertical axis is the amplitude of electric signal (% relative to signal measured in standard AC fluid). Brain sections were placed in flowing standard AC fluid (A), fluid prepared with distilled (B), or micro-clustered water (C). Arrow indicates replacement of standard AC fluid with the test medium prepared with micro-clustered water. Results are averaged for 14 sections from seven rats.

In the first series of experiments the dynamics of induced response amplitude was recorded after replacing standard AC solution with the solution prepared with double distilled water. Immediately after changing the solution, an increase in the induced response amplitude was observed with a maximum at 5 min 128.2% (FIG. 14). A steady decrease in the amplitude was observed, to the point at which after one hour the amplitude decreased to only 31.7% of the initial value.

In the second series of experiments, micro-clustered water was used instead of double distilled water. Immediately after replacing the standard solution with the solution prepared with micro-clustered water, the amplitude of induced response sharply increased, with a maximum of 135.2% reached after 1-3 minutes. Afterwards, the amplitude decreased slightly and after 1 hour it was down to 102%; 2 hours down to 94.8%.

Thus, the results obtained show that replacement of standard AC solution with the solution prepared with micro-clustered water, within experimental error, did not affect the initial amplitude of induced response for 2 hours. Replacement of the standard AC solution with the solution prepared with double distilled water resulted in a decrease to 31.7% (P<0.005) amplitude after 1 hour.

The study was stopped after 2 hours on the micro-clustered solution, as the test unit was out of solution. How long the rat brain tissue would have continued to be viable should be the subject of future studies. At the time the study was stopped the tissues in micro-clustered water still had an average amplitude of 94.8%.

Accordingly, a method of the invention includes stimulating or modulating the growth or activity of cells by contacting the cells for a sufficient period of time with either micro-clustered water or the micro-clustered media compositions of the invention. This method finds utility in using micro-clustered media to enhance the synthesis of compounds or products derived from culture of either animal cells, plant cells, or microorganisms, or from culture of organelles. Typically, the synthesis of compounds or products by these methods involves the preparation of a composition or compound which did not exist in the starting material.

The above examples illustrate that the compositions of the invention are useful in methods of regulating cell metabolism or physiology. Examples of such activities include but are not limited to altering or regulating the differentiation state of said cells, ability of cells to metabolize nutrient materials, cell cycle synchronization or lack thereof, resistance or sensitivity to particular compounds, alteration of intracellular pH. Other methods of using the compositions of the invention find use in mere culturing of cells in a medium, which promotes normal cell growth and division.

Bioprocess Technology; Industrial Product Formation Through Microbial Processes

The micro-cluster water and micro-clustered compositions of the invention are generally useful in bioprocess technology in small, medium and large scale processes, and in methods of production and product recovery or isolation, inoculum and medium preparation, cultivation and downstream processing.

Industrial/pharmaceutical microbiology/biotechnology rely on aqueous compositions, methods of preparing and using them, and the resultant products in the form of small-, medium-, large/macromolecules (Microbial Biotechnology, Fundamentals of Applied Microbiology, Alexander N. Glazer and Hiroshi Nikaido 1995, W. H. Freeman Co.; Pharmaceutical Biotechnology, eds. Daan J. A. Crommelin and Robert D. Sindelar, 1997, Harwood Academic Publishers). The cultivation of cells takes place in vessels containing an appropriate liquid growth medium. Production-scale cultivation is commonly performed in bioreactors which are devices adapted for the growth or propagation of a microorganism or enzyme, or for the synthesis of a composition or compound using a microorganism or enzyme (Ibid, Crommelin, Chapter 3; Glazer at p. 250). Accordingly, the present invention includes the use of micro-clustered compositions in bioreactors in bioprocess technology as described herein.

The compositions of the present invention involve partial or complete substitution of micro-clustered compositions for aqueous compositions heretofore in use by those of skill in the art.

Included in the invention are novel intermediate or final products, which are produced with the micro-clustered compositions, as well as methods of using them Some of the major products dependent on microbial/animal cell/plant cell biotechnology include fermented juices and distilled liquors, cheese, antibiotics, industrial alcohol, high fructose syrups and amino acids, baker's yeast, steroids, vitamins, citric acid, enzymes, hormones, growth factors, vaccines, polysaccharide gums.

Accordingly, the present invention includes micro-clustered compositions and their use in:

1. Production of proteins in bacteria.

2. Production of proteins in yeast.

3. Production of recombinant and synthetic vaccines.

4. Production of microbial insecticides.

5. Production of enzymes

6. Production of microbial polysaccharides and polyesters

7. Production of ethanol

8. Production of amino acids

9. Production of antibiotics

10. Organic synthesis and degradation by enzymes and microbes

11. Environmental applications, including sewage and wastewater microbiology; microbial degradation of xenobiotics; use of microorganisms in mineral recovery, and in removal of heavy metals from aqueous effluents.

Readers of skill in the art to which this invention pertains will understand that the foregoing description of the details of preferred embodiments is not to be construed in any manner as to limit the invention. Such readers will understand that other embodiments may be made which fall within the scope of the invention, which is defined by the following claims and their legal equivalents.

What is claimed is:

1. A culture medium comprising micro-clustered water.

2. A medium comprising micro-clustered water.

3. A culture comprising micro-clustered water.

4. A method of preparing culture medium comprising the step of dissolving or mixing nutrients with micro-clustered water.

5. A method of preparing medium comprising the step of dissolving or mixing one or more of the group of medium ingredients selected from inorganic salts, minerals, carbohydrates, amino acids, vitamins, fatty acids and lipids, proteins and peptides, and serum.

6. A method of preparing a culture comprising the step of contacting cells, tissues, organs, subcellular parts, viruses, bacteriophage, or vectors with a culture medium which comprises micro-clustered water.

7. Use in cell culture of a culture medium which comprises micro-clustered water.

8. Use in cell culture of a medium which comprises micro-clustered water.

9. Use in cell culture of a culture comprising micro-clustered water.

10. Use in microbial biotechnology of one or more of a culture medium which comprises micro-clustered water, a culture which comprises micro-clustered water, and a medium which comprises micro-clustered water.

11. A method of enhancing a cell's viability comprising the step of culturing said cell in one or more of a culture medium which comprises micro-clustered water, a culture which comprises micro-clustered water, and a medium which comprises micro-clustered water.

12. A method of enhancing survivability of a cell, tissue, or organ comprising the step of culturing said cell, tissue, or organ one with one or more of a culture medium which comprises micro-clustered water, a culture which comprises micro-clustered water, and a medium which comprises micro-clustered water.

13. Use in organ, tissue, or cell transplantation of one or more of a culture medium which comprises micro-clustered water, a culture which comprises micro-clustered water, and a medium which comprises micro-clustered water.

14. Use in transfection of one or more of a culture medium which comprises micro-clustered water, a culture which comprises micro-clustered water, and a medium which comprises micro-clustered water.

15. Use in harvesting stem cells of one or more of a culture medium which comprises micro-clustered water, a culture which comprises micro-clustered water, and a medium which comprises micro-clustered water.

16. Use in stem cell biology or cloning of one or more of a culture medium which comprises micro-clustered water, a culture which comprises micro-clustered water, and a medium which comprises micro-clustered water.

17. A kit comprising one or more of a culture medium which comprises micro-clustered water, a culture which comprises micro-clustered water, and a medium which comprises micro-clustered water.
